(19) United States
(12) Patent Application Publication
(10) Pub. No.: US 2003/0092908 A1
(43) Pub. Date: May 15, 2003

(54) Fused heterocyclic inhibitors of phosphodiesterase (PDE) 7

(76) Inventors: William J. Pitts, Newtown, PA (US); Joseph Barbosa, Lambertville, NJ (US); Junqing Guo, Princeton, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/137,508

(22) Filed: May 1, 2002

Related U.S. Application Data
(60) Provisional application No. 60/287,964, filed on May 1, 2001.
(60) Provisional application No. 60/299,287, filed on Jun. 19, 2001.
(60) Provisional application No. 60/368,752, filed on Mar. 29, 2002.

(51) Int. Cl. C07D 487/02

(52) U.S. Cl. 544/254; 544/280

(57) ABSTRACT

Fused heterocylic phosphodiesterase 7 (PDE 7) inhibitors of the following formula

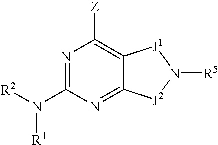

wherein R¹, R², R⁵, Z, J¹ and J² are described herein, and analogs thereof are provided which are useful in treating T-cell mediated diseases.

This application claims priority to U.S. Provisional Application Serial No. 60/287,964 filed May 1, 2001, U.S. Provisional Application Serial No. 60/299,287 filed Jun. 19, 2001, and U.S. Provisional Application Serial No. 60/368,752 filed Mar. 29, 2002. The entirety of each of these applications is incorporated herein by reference.

### FIELD OF THE INVENTION

The present invention relates to fused heterocylic phosphodiesterase 7 (PDE 7) inhibitors, pharmaceutical compositions containing these inhibitiors, and the use of these inhibitors in the treatment of T-cell mediated diseases.

### BACKGROUND OF THE INVENTION

Phosphodiesterases (PDEs) hydrolyze the second messenger molecules cAMP and cGMP to affect cellular signaling. At least 11 families of PDEs exist, some of which (PDE3,4,7,8) are specific for cAMP, and others (PDE5,6,9) for cGMP. Further family members (PDE 1,2,10,11) have dual specificity. A recent publication demonstrated a role for PDE7 in the activation and/or proliferation of T cells(Li, Yee and Beavo, Science 283:848-851, 1999). Resting T lymphocytes express mainly PDE3 and PDE4. However, upon activation, T cells dramatically upregulate PDE7 and appear to rely on this isozyme for regulation of cAMP levels. Removal of the ability to upregulate the production of PDE7 protein by anti-sense oligonucleotides inhibited the proliferation and IL-2 production along with the maintenance of high concentrations of intracellular cAMP in CD3xCD28 stimulated T cells.

A PDE7 inhibitor is defined herein as a compound for which the IC₅₀ of the compound in a PDE7 inhibition assay is less than 20 micromolar (preferably less than 10 micromolar, more preferably less than 5 micromolar, most preferably less than 1 micromolar). The PDE7 IC₅₀ of a selective PDE7 inhibitor should be less than one-tenth the IC50 of said compound in all of the following PDE assays: PDE1, PDE3 and PDE4 (more preferably the PDE7 IC₅₀ of a selective PDE7 inhibitor should be less than one-twentieth the IC₅₀ of said compound in the following PDE assays: PDE1 and PDE3, most preferably the PDE7 C₅₀ of a selective PDE7 inhibitor should be less than one-hundreth the IC₅₀ of said compound in a PDE3 assay).

Several isoforms of PDE1 have been identified and are distributed in heart, lung, and kidney tissue, as well as in circulating blood cells and smooth muscle cells. PDE1 inhibitors have demonstrated potent vasodilator activity. Such activity would represent an undesirable side effect in a therapeutic agent with the utilities listed in this patent for a PDE7 inhibitor. The PDE3 family of enzymes are distributed in several tissues including the heart liver, and platelets. PDE3 inhibitors have demonstrated potent cardiac iotropic activity. Such activity would represent an undesirable side effect in a therapeutic agent with the utilities listed in this patent for a PDE7 inhibitor. Several isoforms of PDE4 exist, and these are expressed in a wide variety of tissues including heart, kidney, brain, the gastrointestinal track and circulating blood cells. PDE4 inhibitors have demonstrated clinical utility for COPD, and have also been suggested to have utility for rheumatoid arthritis, and multiple sclerosis, and to possess anti-inflammatory activity. The utility of PDE4 inhibitors has been limited to some extent by their propensity to cause emesis. As such there are circumstances where it would be desirable to develop PDE7 inhibitors, which have a degree of selectivity against PDE. A selective inhibitor of PDE7 is expected to have broad application as an immunosuppressant in T cell-mediated diseases. PDE7 inhibitors will act at a different stage of the T cell signaling process compared to current immunosuppressants by inhibiting a very early stage of the T cell activation cascade. A selective inhibitor of PDE7 is also expected to have a decreased potential for clinically significant side effects compared to current immunosuppressants, therefore the primary disease indications are solid organ transplantation (SOT) and rheumatoid arthritis. Additional indications may include IBD, psoriasis, asthma and lupus.

A dual PDE7-PDE4 inhibitor (PDE4/7 or PDE7/4) is defined herein as any compound which has an IC50 in both a PDE7 and a PDE4 inhibition assay of less than 20 micromolar (preferably less than 10 micromolar, and more preferably less than 5 micromolar and most preferably less than 1 micromolar), and an IC50 in a PDE3 inhibition assay which is at least 10 times higher than the IC50 of the compound in the PDE7 assay (more preferably at least 20 times higher than the IC50 of the compound in the PDE7 assay, and most preferably at least 100 times higher than the IC50 of the compound in the PDE7 assay). A dual PDE417 inhibitor should have a ratio of inhibition or PDE7 IC50 divided by PDE4 IC50 of between one-tenth and 100. Inhibitors that exhibit such a ratio of inhibition include those that inhibit PDE3, PDE4 and PDE7 as described above, and further inhibit PDE1 at an IC50 at least 10 times higher than the IC50 of the compound in a PDE7 assay (more preferably at least 20 times higher than the IC50 of the compound in the PDE7 assay, and most preferably at least 100 times higher than the IC50 of the compound in the PDE7 assay). Preferred dual PDE7-PDE4 inhibitors further include those compounds that inhibit PDE3, PDE4 and PDE7 as described above, and further suppress both T cell proliferation, and TNF-alpha secretion from either THP-1 monocytes or human peripheral blood mononuclear cells at a level of less than 20 micromolar.

“Leukocyte activation” is defined herein as any or all of leukocyte (T cell, monocyte macrophage, neutrophil etc.) cell proliferation, cytokine production, adhesion protein expression, and production of inflammatory mediators. This is mediated in part by the action of PDE4 and/or PDE7 depending on the particular leukocyte under consideration.

Examples of leukocyte activation associated or leukocyte activation mediated disorders include transplant rejection, graph verses host disease, and autoimmune disorders, such as rheumatoid arthritis, multiple sclerosis, juvenile diabetes, COPD, asthma, and inflammatory bowel disease, T-cell mediated hypersensitivity diseases, ischemic or reperfusion injury, and T-cell proliferative disorders.

Dual PDE4/7 inhibitors would be expected to block the T cell component of a disease as well as possess anti-inflammatory activity. Thus a dual PDE4/7 inhibitor which is not significantly limited by emesis, may be more effective than either a selective PDE4 inhibitor or a selective PDE7 inhibitor in a variety of disease states such as rheumatoid arthritis, asthma, COPD and multiple sclerosis.

Development of either selective PDE7 inhibitors, or dual PDE7-PDE4 inhibitors will yield novel classes of therapeutics and have a novel mechanism of action by maintaining high levels of intracellular cAMP. These inhibitors would target a major unmet medical need in an area where current therapies possess significant toxicity.

Two PDE7 genes have been identified. PDE7A (EC 3.1.4.17) has two isoforms generated by alternate splicing; PDE7A1 restricted mainly to T cells and the brain, and PDE7A2 for which mRNA is expressed in a number of cell types including muscle cells. The isoforms have different sequence at the amino termini, and it is thought that this portion of each molecule is likely to be important for cellular localization of the enzyme. However, the catalytic domain of each PDE7A enzyme is identical (Han, P., Zhu,X. and Michaeli,T. Alternative splicing of the high affinity cAMP-specific phosphodiesterase (PDE7A) mRNA in human skeletal muscle and heart. J. Biol. Chem. 272 (26), 16152-16157 (1997)). Although abundant PDE7A2 mRNA has been identified, the presence of active enzyme in tissues is controversial, as no convincing data shows PDE7A2 protein in situ in the adult. PDE7B (EC 3.1.4.17), a second PDE7 gene family member, has approximately 70% homology to PDE7A in the enzymatic core (Sasaki, T., Kotera,J., Yuasa,K. and Omori,K. Identification of human PDE7B, a cAMP-specific phosphodiesterase Biochem. Biophys. Res. Commun. 271 (3), 575-583 (2000)). Two patents from Cold Spring Harbor Labs (U.S. Pat. No. 5,527,896 and U.S. Pat. No. 5,977,305) cover the methods of preparation and use of recombinant PDE7A protein. A recent publication describes moderately active PDE7 inhibitors (J. Med Chem. Vol. 43, 683 (2000)). WO 00/68230 discloses certain 1,9 dihydropurin-6-ones derivatives as PDE7 inhibitors.

### SUMMARY OF THE INVENTION

The present invention provides novel fused heterocyclic compounds of the following formula (I), their enantiomers, diastereomers, and pharmaceutically acceptable salts, prodrugs and solvates thereof, for use as PDE7 inhibitors:

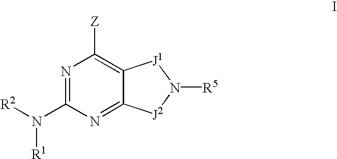

wherein:

R¹ is hydrogen or alkyl;

R²is

(a) heteroaryl, or heterocyclo, either of which may be optionally substituted with one to three groups T¹, T², T³;

(b) aryl substituted with one to three groups T¹, T², T³ provided that at least one of T¹, T², T³ is other than H; or

(c) aryl fused to a heteroaryl or heterocyclo ring wherein the combined ring system may be optionally substituted with one to three groups T¹, T², T³;

Z is NR³R⁴, NR³SO₂R^{4a}, OR⁴, SR⁴, haloalkyl, or halogen;

R³ and R⁴ are independently H, alkyl, alkenyl, aryl, (aryl)alkyl, heteroaryl, (heteroaryl)alkyl, cycloalkyl, (cycloalkyl)alkyl, heterocylo or (heterocyclo)alkyl any of which may be optionally independently substituted where valance allows with one to three groups T^{1a}, T^{2a} or T^{3a};

or R³ and R⁴ may be taken together with the nitrogen atom to which they are attached to form a heterocyclo or heteroaryl ring optionally independently substituted where valance allows with one to three groups T^{1a}, T^{2a} or T^{3a};

R^{4a} is alkyl, alkenyl, aryl, (aryl)alkyl, heteroaryl, (heteroaryl)alkyl, cycloalkyl, (cycloalkyl)alkyl, heterocylo or (heterocyclo)alkyl any of which may be optionally independently substituted where valance allows with one to three groups T^{1a}, T^{2a} or T^{3a};

R^{3b} and R^{4b} are independently H, alkyl, alkenyl, aryl, (aryl)alkyl, heteroaryl, (heteroaryl)alkyl, cycloalkyl, (cycloalkyl)alkyl, heterocylo or (heterocyclo)alkyl;

R⁵ is

(a) hydrogen, or cyano;

(b) alkyl, alkenyl, alkynyl, cycloalkyl, (cycloalkyl)alkyl, aryl, (aryl)alkyl, heterocyclo, (heterocyclo)alkyl, heteroaryl or (heteroaryl)alkyl, any of which may be optionally independently substituted where valance allows with one to three groups T^{1b}, T^{2b} or T^{3b}; or

(c) —C(O)R⁶, —C(O)OR⁶, —C(O)—C(O)OR 6, or —SO₂R^{6a};

R⁶ is H, alkyl, alkenyl, —NR^{3b}R^{4b}, heterocylco, (heterocyclo)alkyl, (hydroxy)alkyl, (alkoxy)alkyl, (aryloxy)alkyl, (NR^{3b}R^{4b})alkyl, heteroaryl, aryl or (aryl)alkyl, any of which may be optionally independently substituted where valance allows with one to three groups T^{1b}, T^{2b} or T^{3b};

R^{6a}, is alkyl, alkenyl, —NR^{3b}R^{4b}, heterocylco, (heterocyclo)alkyl, (hydroxy)alkyl, (alkoxy)alkyl, (aryloxy)alkyl, (NR^{3b}R^{4b})alkyl, heteroaryl, aryl or (aryl)alkyl, any of which may be optionally independently substituted where valance allows with one to three groups T^{1b}, T^{2b} or T^{3b};

J¹ and J² are independently optionally substituted C₁₋₃ alkylene, provided that J¹ and J² are not both greater than C₂ alkylene;

T^{1-1b}, T^{2-2b} and T^{3-3b} are are each independently

(1) hydrogen or T⁶, where T⁶ is

(i) alkyl, (hydroxy)alkyl, (alkoxy)alkyl, alkenyl, alkynyl, cycloalkyl, (cycloalkyl)alkyl, cycloalkenyl, (cycloalkenyl)alkyl, aryl, (aryl)alkyl, heterocyclo, (heterocylco)alkyl, heteroaryl, or (heteroaryl)alkyl;

(ii) (ii) a group (i) which is itself substituted by one or more of the same or different groups (i); or

(iii) (iii) a group (i) or (ii) which is independently substituted by one or more (preferably 1 to 3) of the following groups (2) to (13) of the definition of T^{1-1b}, T^{2-2b} and T^{3-3b},

(2) —OH or —OT⁶,

(3) —SH or —ST⁶,

(4) —C(O)ₜH, —C(O)ₜT⁶, or —O—C(O)T⁶, where t is 1 or 2;

(5) —SO₃H, —S(O)ₜT⁶, or S(O),N(T⁹)T⁶,

(6) halo,

(7) cyano,

(8) nitro,

(9) —T⁴—NT⁷T⁸,

(10) —T—N(T⁹)—T⁵—NT⁷T,

(11) —T⁴—N(T¹⁰)—T⁵-T⁶,

(12) —T⁴—N(T¹⁰)—T⁵—H,

(13) oxo,

T⁴ and T⁵ are each independently

(1) a single bond,

(2) —T¹—S(O)ₜ—T¹²—,

(3) —T¹¹—C(O)—T¹²—,

(4) —T¹¹—C(S)—T¹²—,

(5) —T¹¹—O—T¹²—,

(6) —T¹¹—S—T¹²—,

(7) —T¹¹—O—C(O)—T¹²—,

(8) —T¹¹—C(O)—O—T¹²—,

(9) —T¹¹—C(═NT^{9a})—T¹²—, or

(10) —T¹¹—C(O)—C(O)—T¹²—

T⁷, T⁸, T⁹, T^{9a} and T¹⁰

(1) are each independently hydrogen or a group provided in the definition of T⁶, or

(2) T⁷ and T⁸ may together be alkylene or alkenylene, completing a 3- to 8-membered saturated or unsaturated ring together with the atoms to which they are attached, which ring is unsubstituted or substituted with one or more groups listed in the description of T^{1-1b}, T^{2-2b} and T^{3-3b}, or

(3) T⁷ or T⁸, together with T⁹, may be alkylene or alkenylene completing a 3- to 8-membered saturated or unsaturated ring together with the nitrogen atoms to which they are attached, which ring is unsubstituted or substituted with one or more groups listed in the description of T^{1-1b}, T^{2-2b} and T^{3-3b}, or

(4) T and T⁸ or T⁹ and T¹⁰ together with the nitrogen atom to which they are attached may combine to form a group —N═CT¹³T¹⁴ where T¹³ and T¹⁴ are each independently H or a group provided in the definition of T6; and

T¹¹ and T¹² are each independently

(1) a single bond,

(2) alkylene,

(3) alkenylene, or

(4) alkynylene.

Preferred compounds within the scope of the present invention include compounds wherein the subsitutents R¹, R², Z, J¹, J² and R⁵ are selected from the following:

R¹ is H;

R² is

(a) heteroaryl (more preferably thiazolyl or oxazolyl) optionally substituted with one to three groups T¹, T², T³, preferably including H, alkyl, haloalkyl, halo, heteroaryl, C(O)ₜT⁶, OT⁶, —T⁴NT⁷T⁸

(b) aryl substituted with one to three groups T¹, T², T³ (preferably including heteroaryl (preferably, imidazolyl, oxazolyl, or thiazolyl any of which may be further optionally substituted), cyano, C(O)ₜT⁶, S(O)ₜN(T⁹)T⁶, halo alkyl, and haloalkyl); or

(c) aryl fused to a heteroaryl ring (e.g., quinolyl bound through the aryl ring (especially quinol-6-yl), quinazolinyl bound through the aryl ring (especially quinazolin-6-yl), cinnolinyl bound through the aryl ring (especially cinnolin-6-yl), isoqinolinyl bound through the aryl ring (especially isoquinol-6-yl), and phthalazinyl bound through the aryl ring (especially phthalazin-6-yl)) wherein the combined ring system may be optionally substituted with one to three groups T¹, T², T³;

Z is NR³R⁴, or OR⁴;

R³ is H or alkyl, cycloalkyl,

R⁴ is alkyl optionally independently substituted with one to three groups T^{1a}, T^{2a} or T^{3a}, or (aryl)alkyl optionally independently substituted with one to three groups T^{1a}, T^{2a} or T^{3a}.(especially where the aryl group is independently substituted with one or more OT⁶, S(O)ₜT⁶ or S(O)ₜN(T⁹)T⁶);

or R³ and R⁴ may be taken together with the nitrogen atom to which they are attached to form a heterocyclo ring (especially including piperidyl, piperazinyl, and morpholinyl) optionally independently substituted with one to three groups T^{1a}, T^{2a} or T^{3a} (especially including hydroxy, oxo, and —C(O)ₜT⁶);

R⁵ is

(a) hydrogen, or cyano;

(b) alkyl, alkenyl, alkynyl, cycloalkyl, (cycloalkyl)alkyl, aryl, (aryl)alkyl, heterocyclo, (heterocyclo)alkyl, heteroaryl (especially including pyridyl, furanyl, thienyl, and thiazoly) or (heteroaryl)alkyl, any of which may be optionally independently substituted one to three groups T^{1b}, T^{2b} or T^{3b} (especially including cyano, —OT⁶, —C(O)ₜT⁶ and —S(O)ₜT⁶); or (c) —C(O)R⁶, —C(O)OR⁶, —C(O)—C(O)OR⁶, or —SO₂R^{6a};

R⁶ is H, alkyl, alkenyl, —NR^{3b}R^{4b}, heterocylco (especially including morpholinyl, piperazinyl, and tetrahydrofuranyl), (heterocyclo)alkyl, (hydroxy)alkyl, (alkoxy)alkyl, (aryloxy)alkyl, (NR^{3b}R^{4b})alkyl, heteroaryl, (heteroaryl)alkyl, aryl or (aryl)alkyl, any of which may be optionally independently substituted where valance allows with one to three groups T^{1b}, T^{2b} or T^{3b} (especially where T^{1b}, T^{2b} or T^{3b} include alkyl, —C(O)ₜH, —C(O)ₜT⁶, —OC(O)T⁶, —OH, —OT⁶, and —S(O)ₜT⁶);

R^{6a} is alkyl, alkenyl, —NR^{3b}R^{4b}, heterocylco (especially including morpholinyl, piperazinyl, and tetrahydrofuranyl), (heterocyclo)alkyl, (hydroxy)alkyl, (alkoxy)alkyl, (aryloxy)alkyl, (NR^{3b}R^{4b})alkyl, heteroaryl (especially including pyridyl, furanyl, thienyl, and thiazoly), (heteroaryl)alkyl, aryl or (aryl)alkyl, any of which may be optionally independently substituted where valance allows with one to three groups T^{1b}, T^{2b} or T^{3b} (especially where T^{1b}, T^{2b} or T^{3b} include alkyl, —C(O)ₜH, —C(O)ₜT⁶, —OC(O)T⁶, —OH, —OT⁶, and —S(O)ₜT⁶); and

J¹ and J² are independently optionally substituted C₁₋₃ alkylene, provided that J¹ and J² are not both greater than C₂ alkylene.

More preferred compounds within the scope of the present invention include compounds wherein the subsitutents R¹, R², Z, J¹, J² and R⁵ are selected from the following:

R¹ is H;

R² is

(a) thiazolyl optionally substituted with one to three groups T¹, T², T³, preferably including H, alkyl, haloalkyl, halo, heteroaryl, C(O)ₜT⁶, OT⁶, —T⁴NT⁷T⁸

(b) phenyl substituted at the para position with an electon-donar group T¹ (such as heteroaryl (preferably, imidazolyl, oxazolyl, or thiazolyl any of which may be further optionally substituted), cyano, C(O)ₜT⁶, or S(O)ₜN(T⁹)T⁶) and optionally further substituted with groups T and T³ (including cyano, C(O)ₜT⁶, S(O)ₜN(T⁹)T⁶, halo alkyl, and haloalkyl); or

(c) aryl fused to a heteroaryl ring (e.g., quinolyl bound through the aryl ring (especially quinol-6-yl), quinazolinyl bound through the aryl ring (especially quinazolin-6-yl), cinnolinyl bound through the aryl ring (especially cinnolin-6-yl), isoqinolinyl bound through the aryl ring (especially isoquinol-6-yl), and phthalazinyl bound through the aryl ring (especially phthalazin-6-yl)) wherein the combined ring system may be optionally substituted with one to three groups T¹, T², T³;

Z is NR³R⁴

R³ is H or alkyl, cycloalkyl,

R⁴ is (aryl)alkyl optionally independently substituted with one to three groups T^{1a}, T^{2a} or T^{3a}.(especially where the aryl group is independently substituted with one or more OT⁶, S(O)ₜT⁶ or S(O)ₜN(T⁹)T⁶);

or R³ and R⁴ may be taken together with the nitrogen atom to which they are attached to form a heterocyclo ring (especially including piperidyl, piperazinyl, and morpholinyl) optionally independently substituted with one to three groups T^{1a}, T^{2a} or T^{3a} (especially including hydroxy, oxo, and —C(O)ₜT);

R⁵ is

(a) hydrogen, or cyano;

(b) alkyl, alkenyl, (cycloalkyl)alkyl, (aryl)alkyl, or (heteroaryl)alkyl (where the heteroaryl groups include pyridyl, furanyl, thienyl, and thiazoly), any of which may be optionally independently substituted one to three groups T^{1b}, T^{2b} or T^{3b} (especially including cyano, —OT⁶, and —S(O)ₜT⁶); or

(c) —C(O)R⁶, —C(O)OR⁶, —C(O)—C(O)OR⁶, or SO₂R^{6a};

R⁶ is H, alkyl, alkenyl, —NR^{3b}R^{4b}, heterocylco (especially including morpholinyl, piperazinyl, and tetrahydrofuranyl), (heterocyclo)alkyl, (hydroxy)alkyl, (alkoxy)alkyl, (aryloxy)alkyl, (NR^{3b}R^{4b})alkyl, any of which may be optionally independently substituted where valance allows with one to three groups T^{1b}, T^{2b} or T^{3b} (especially where T^{1b}, T^{2b} or T^{3b} include alkyl, —C(O)ₜH, —C(O)ₜT -OC(O)T⁶, —OH, —OT⁶, and —S(O)ₜT⁶);

R^{6a} is H, alkyl, alkenyl, —NR^{3b}R^{4b}, heterocylco (especially including morpholinyl, piperazinyl, and tetrahydrofuranyl), (heterocyclo)alkyl, (hydroxy)alkyl, (alkoxy)alkyl, (aryloxy)alkyl, (NR^{3b}R^{4b})alkyl, any of which may be optionally independently substituted where valance allows with one to three groups T^{1b}, T^{2b} or T^{3b} (especially where T^{1b}, T^{2b} or T^{3b} include alkyl, —C(O)ₜH, —C(O)ₜT⁶, —OC(O)T⁶, —OH, —OT⁶, and —S(O)ₜT⁶); and

J¹ and J² are independently optionally substituted C₁₋₃ alkylene, provided that J¹ and J² are not both greater than C₂ alkylene.

Preferred compounds of the present invention include compounds of formula (IIa), and formula (IIb)

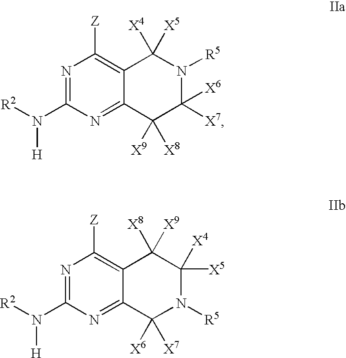

wherein:

R² is chosen from:

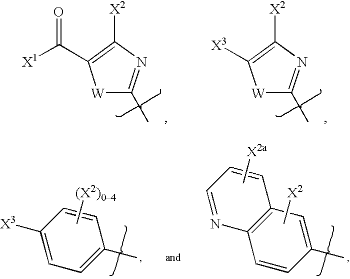

wherein:

W is O or S, more preferably S;

X¹ is NHT⁸ or OT⁶;

X² and X^{2a} are independently hydrogen, halo, OT⁶, or alkyl; and

X³ is heteroaryl (preferably, imidazolyl, oxazolyl, or thiazolyl any of which may be further optionally substituted), cyano, C(O)ₜT⁶, or S(O)ₜN(T⁹)T⁶;

X⁴, X⁵, X⁶ and X⁷ are independently chosen from hydrogen, T⁶, OT⁶, or NT⁷T⁸, or X⁴ and X⁵ or X⁶ and X⁷ may be taken together to be a carbonyl group; and

X⁸ and X⁹ are independently chosen from hydrogen, T⁶, OT⁶, or NT⁷T⁸.

Preferred compounds of the present invention include compounds of formulas (IIIa), (IIIb) and (IIIc)

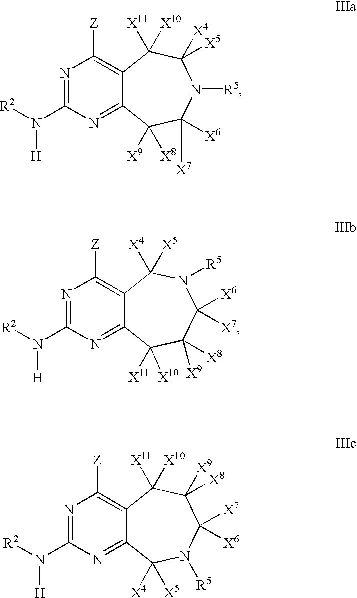

wherein:

R² is chosen from:

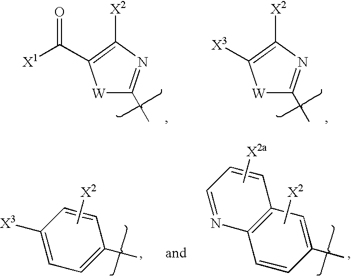

wherein:

W is O or S, more preferably S;

X¹ is NHT⁸ or OT⁶;

X² is hydrogen, halo, OT⁶, or alkyl;

X³ is heteroaryl (preferably, imidazolyl, oxazolyl, or thiazolyl any of which may be further optionally substituted), cyano, C(O)ₜT⁶, or S(O)ₜN(T⁹)T⁶;

X⁴, X⁵, X⁶ and X⁷ are independently chosen from hydrogen, T⁶, OT⁶, or NT⁷T⁸, or X⁴ and X⁵, or X⁶ and X⁷ may be taken together to be a carbonyl group; and

X⁸, X⁹X¹⁰, and X¹¹ are independently chosen from hydrogen, T⁶, OT⁶, or NT⁷T⁸.

Preferred compounds of the present invention include compounds of formula (IV)

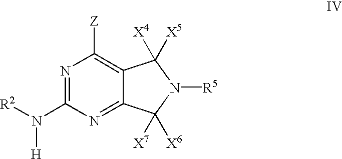

wherein:

R² is chosen from:

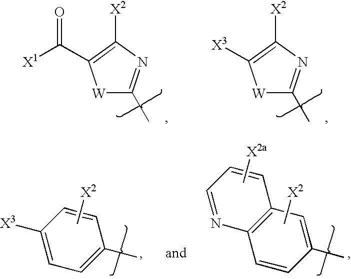

wherein:

W is O or S, more preferably S;

X¹ is NHT⁸ or OT⁶.

X² is hydrogen, halo, OT⁶, or alkyl.

X³ is heteroaryl (preferably, imidazolyl, oxazolyl, or thiazolyl any of which may be further optionally substituted), cyano, C(O)ₜT⁶, or S(O)ₜN(T⁹)T⁶;

X⁴, X⁵, X⁶ and X⁷ are independently chosen from hydrogen, T⁶, OT⁶, NT⁷T⁸, or X and

X⁵, or X⁶ and X⁷ may be taken together to be a carbonyl group.

Compounds within the scope of formula I include compounds that are dual PDE7-PDE4 inhibitors. Dual PDE7-PDE4 compounds include compounds of formula V

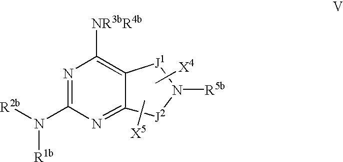

wherein

R^{1b} is H or alkyl;

R^{2b} is optionally substituted heteroaryl;

R^{3b} is H or alkyl;

R^{4b} is optionally substituted (aryl)alkyl;

R^{5b} is H, alkyl, or —C(O)—(CH₂)ᵥ—O—Y—R^{6b}, where Y is a bond or —C(O)—, R^{6b} is hydrogen or alkyl, and v is an integer from 0 to 2;

J¹ and J² are independently optionally substituted C₁₋₃ alkylene, provided that J¹ and J² are not both greater than C₂ alkylene;

X⁴ and X⁵ are optional substituents bonded to any available carbon atom in one or both of J¹ and J², independently selected from hydrogen, OR⁷, NR⁸R⁹, alkyl, substituted alkyl, alkenyl, substituted alkenyl, alkynyl, substituted alkynyl, cycloalkyl, substituted cycloalkyl, aryl, substituted aryl, heterocycloalkyl, or heteroaryl;

R⁷ is hydrogen, alkyl, substituted alkyl, alkenyl, alkynyl, cycloalkyl, substituted cycloalkyl, C(O)alkyl, C(O)substituted alkyl, C(O)cycloalkyl, C(O) substituted cycloalkyl, C(O)aryl, C(O)substituted aryl, C(O)Oalkyl, C(O)Osubstituted alkyl, C(O)heterocycloalkyl, C(O)heteroaryl, aryl, substituted aryl, heterocycloalkyl and heteroaryl; and

R⁸ and R⁹ are independently selected from the group consisting of hydrogen, alkyl, substituted alkyl, cycloalkyl, substituted cycloalkyl, alkenyl, alkynyl, C(O)alkyl, C(O)substituted alkyl, C(O)cycloalkyl, C(O)substituted cycloalkyl, C(O)aryl, C(O)substituted aryl, C(O)Oalkyl, C(O)Osubstituted alkyl, C(O)heterocycloalkyl, C(O)heteroaryl, S(O)₂alkyl, S(O)₂substituted alkyl, S(O)₂cycloalkyl, S(O)₂substituted cycloalkyl, S(O)₂aryl, S(O)₂substituted aryl, S(O)₂heterocycloalkyl, S(O)₂heteroaryl, aryl, substituted aryl, heterocycloalkyl, and heteroaryl, or R₈ and R₉ taken together with the nitrogen atom to which they are attached complete an optionally substituted heterocycloalkyl or heteroaryl ring.

Preferred compounds within the scope of formula V include compounds of formula Va and Vb

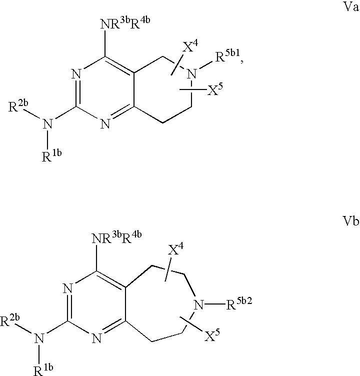

wherein

R^{1b}, R^{2b}, R^{3b}, R⁴b, X⁴ and X⁵ are as defined above;

R^{5b1} is H or alkyl; and

R^{5b2} is —C(O)—(CH₂)ᵥ—O—Y—R^{6b}, where Y is a bond or —C(O)—, R is hydrogen or alkyl, and v is an integer from 0 to 2;

Preferred compounds within Formula V are those wherein:

R^{1b} is H;

R^{2b} is thiazolyl, oxazolyl, or isoxozolyl (preferably thiazolyl) any of which may be optionally substituted (preferably with one or more alkyl, or alkoxycarbonyl groups);

R^{3b} is H;

R^{4b} is optionally substituted (pheny)alkyl, (preferably substituted with one or more group of the formula —SO₂R^{8b} where R^{8b} is alkyl, amino, alkylamino or dialkylamino);

R^{5b} is alkyl, or —C(O)—(CH₂)ᵥ—O—Y—R^{6b}, where Y is a bond or —C(O)—, R^{6b} is hydrogen or alkyl, and v is 1;

J¹ is an alkylene group of 1 or 2 carbon atoms;

J² is an alkylene group of 2 carbon atoms; and

X⁴ and X⁵ are each H.

More preferred compounds within Formula V are those wherein

R^{1b} is H;

R^{2b} is

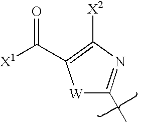

where W is O or S (preferably S), X¹ is alkoxy, and X² is alkyl;

R^{3b} is H;

R^{4b} is (pheny)alkyl substituted with one or more group of the formula —SO₂R^{8b} where R^{8b} is alkyl, or amino;

R^{5b} is alkyl, or —C(O)—(CH₂)ᵥ—O—Y—R^{6b}, where Y is a bond or —C(O)—, R^{6b} is hydrogen or alkyl, and v is 1;

J¹ is an alkylene group of 1 or 2 carbon atoms; ^{J}² is an alkylene group of 2 carbon atoms; and

X⁴ and X⁵ are each H.

Preferred compounds within the scope of Formula V include:

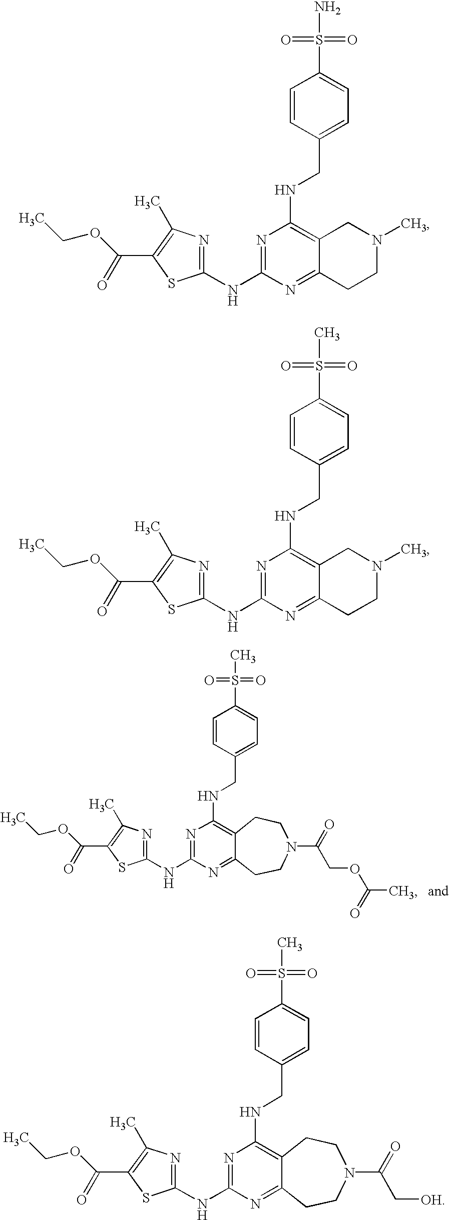

The following are definitions of the terms as used throughout this specification and claims. The initial definition provided for a group or term herein applies to that group or term throughout the present specification, individually or as part of another group, unless otherwise indicated.

The terms “alk” or “alkyl” refer to straight or branched chain hydrocarbon groups having 1 to 12 carbon atoms, preferably 1 to 8 carbon atoms, such as methyl, ethyl, n-propyl, i-propyl, n-butyl, i-butyl, t-butyl, pentyl, hexyl, heptyl, octyl, etc. Lower alkyl groups, that is, alkyl groups of 1 to 6 carbon atoms, are generally most preferred.

The term “substituted alkyl” refers to alkyl groups substituted with one or more groups listed in the definition of T¹, T² and T³, preferably selected from halo, cyano, O—R₇, S—R₇, NR₈R₉, nitro, cycloalkyl, substituted cycloalkyl, oxo, aryl, substituted aryl, heterocyclo, heteroaryl, CO₂R₇, S(O)R₇, SO₂R₇, SO₃R₇, SO₂NR₈R₉, C(O)NR₈R₉, C(O)alkyl, and C(O)H.

The term “alkylene” refers to a straight chain bridge of 1 to 4 carbon atoms connected by single bonds (e.g., —(CH₂)ₓ— wherein x is 1 to 5), which may be substituted with one or more groups listed in the definition of T¹, T² and T³.

The term “alkenyl” refers to straight or branched chain hydrocarbon groups having 2 to 12 carbon atoms, preferably 2 to 4 carbon atoms, and at least one double carbon to carbon bond (either cis or trans), such as ethenyl.

The term “.substituted alkenyl” refers to an alkenyl group as defined above substituted with one or more groups listed in the definition of T¹, T² and T³, preferably selected from halo, cyano, O—R₇, S—R₇, NR₈R₉, nitro, cycloalkyl, substituted cycloalkyl, oxo, aryl, substituted aryl, heterocyclo, heteroaryl, CO₂R₇, S(O)R₇, SO₂R₇, SO₃R₇, SO₂NR₈R₉, C(O)NR₈R₉, C(O)alkyl, and C(O)H.

The term “alkynyl” refers to straight or branched chain hydrocarbon group having 2 to 12 carbon atoms and one, two or three triple bonds, preferably 2 to 6 carbon atoms and one triple bond.

The term “substituted alkynyl” refers to an alkynyl group as defined above substituted with one or more groups listed in the definition of T¹, T² and T³, preferably selected from halo, cyano, O—R₇, S—R₇, NR₈R₉, nitro, cycloalkyl, substituted cycloalkyl, oxo, aryl, substituted aryl, heterocyclo, heteroaryl, CO₂R₇, S(O)R₇, SO₂R₇, SO₃R₇, SO₂NR₈R₉, C(O)NR₈R₉, C(O)alkyl, and C(O)H.

The term “halo” refers to chloro, bromo, fluoro, and iodo.

The term “cycloalkyl” refers to saturated and partially unsaturated (containing 1 or 2 double bonds) cyclic hydrocarbon groups containing 1 to 3 rings, including monocyclicalkyl, bicyclicalkyl and tricyclicalkyl, containing a total of 3 to 20 carbons forming the rings, preferably 3 to 7 carbons, forming the ring and which may be fused to 1 or 2 aromatic or heterocyclo rings, which include cyclopropyl, cyclobutyl, cyclopentyl, cyclohexyl, cycloheptyl, cyclooctyl, cyclodecyl, cyclododecyl, cyclohexenyl,

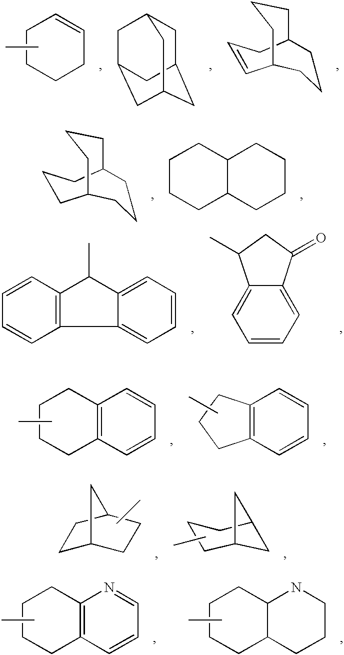

and the like.

The term “¹substituted cycloalkyl” refers to such cycloalkyl group as defined above substituted with one or more groups listed in the definition of T¹, T² and T³, preferably selected from halogen, nitro, alkyl, substituted alkyl, alkenyl, cyano, cycloalkyl, substituted cycloalkyl, aryl, substituted aryl, heterocyclo, heteroaryl, oxo, OR₇, CO₂R₇, C(O)NR₈R₉, OC(O)R₇, OC(O)OR₇, OC(O)NR₈R₉, OCH₂CO₂R₇, C(O)R₇, NR₈R₉, NR₁₀C(O)R₇, NR₁₀C(O)OR₇, NR₁₀C(O)C(O)OR₇, NR₁₀C(O)C(O)NR₈R₉, NR₁₀C(O)C(O)alkyl, NR₁₀C(NCN)OR₇, NR₁₀C(O)NR₈R₉, NR₁₀C(NCN)NR₈R₉, NR₁₀C(NR₁₁)NR₈R₉, NR₁₀SO₂NR₈R₉, NR₁₀SO₂R₇, SR₇, S(O)R₇, SO₂R₇, SO₃R₇, SO₂NR₈R₉, NHOR₇, NR₁₀NR₈R₉, N(COR₇)OR₁₀, N(CO₂R₇)OR₁₀, C(O)NR₁₀(CR₁₂R₁₃)ᵣR₇, CO(CR₁₂R₁₃)ₚO(CR₁₄R₁₅)_{q}CO₂R₇, CO(CR₁₂R₁₃)ᵣOR₇, CO(CR₁₂R₁₃)ₚO(CR₁₄R₁₅)_{q}R₇, CO(CR₁₂R₁₃)ᵣNR₈R₉, OC(O)O(CR₁₂R₁₃)ₘNR₈R₉, OC(O)N(CR₁₂R₁₃)ᵣR₇, O(CR₁₂R₁₃)ₘNR₈R₉, NR₁₀C(O)(CR₁₂R₁₃)ᵣR₇, NR₁₀C(O)(CR₁₂R₁₃)ᵣOR₇, NR₁₀C(═NC)(CR₁₂R₁₃)ᵣR₇, NR₁₀CO(CR₁₂R₁₃)ᵣNR₈R₉, NR₁₀(CR₁₂R₁₃)ₘOR₇, NR₁₀(CR₁₂R₁₃)ᵣCO₂R₇, NR₁₀(CR₁₂R₁₃)ₘNR₈R₉, NR₁₀(CR₁₂R₁₃)ₙSO₂(CR₁₄R₁₅)_{q}R₇, CONR₁₀(CR₁₂R₁₃)ₙSO₂(CR₁₄R₁₅)_{q}R₇, SO₂NR₁₀(CR₁₂R₁₃)ₙCO(CR₁₄R₁₅)_{q}R₇, and SO₂NR₁₀(CR₁₂R₁₃)ₘOR₇.

The terms “ar” or “aryl” refer to aromatic homocyclic (i.e., hydrocarbon) mono-, bi- or tricyclic ring-containing groups preferably having 6 to 12 members such as phenyl, naphthyl and biphenyl, as well as such rings fused to a cycloalkyl, cycloalkenyl, heterocyclo, or heteroaryl ring. Examples include:

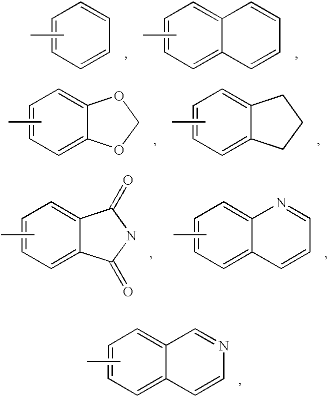

and the like.

The term “substituted aryl” refers to such aryl groups as defined above substituted with one or more groups listed in the definition of T¹, T² and T³, preferably selected from halogen, nitro, alkyl, substituted alkyl, alkenyl, cyano, cycloalkyl, substituted cycloalkyl, aryl, substituted aryl, heterocyclo, heteroaryl, OR₇, CO₂R₇, C(O)NR₈R₉, OC(O)R₇, OC(O)OR₇, OC(O)NR₈R₉, OCH₂CO₂R₇, C(O)R₇, NR₈R₉, NR₁₀C(O)R₇, NR₁₀C(O)OR₇, NR₁₀C(O)C(O)OR₇, NR₁₀C(O)C(O)NR₈R₉, NR₁₀C(O)C(O)alkyl, NR₁₀C(NCN)OR₇, NR₁₀C(O)NR₈R₉, NR₁OC(NCN)NR₈R₉, NR₁₀C(NR₁₁)NR₈R₉, NR₁₀SO₂NR₈R₉, NR₁₀SO₂R₇, SR₇, S(O)R₇, SO₂R₇, SO₃R₇, SO₂NR₈R₉, NHOR₇, NR₁₀NR₈R₉, N(COR₇)OR₁₀, N(CO₂R₇)OR₁₀, C(O)NR₁₀(CR₁₂R₁₃)ᵣR₇, CO(CR₁₂R₁₃)ₚO(CR₁₄R₁₅)_{q}CO₂R₇, CO(CR₁₂R₁₃)ᵣOR₇, CO(CR₁₂R₁₃)ₚO(CR₁₄R₁₅)_{q}R₇, CO(CR₁₂R₁₃)ᵣNR₈R₉, OC(O)O(CR₁₂R₁₃)ₘNR₈R₉, OC(O)N(CR₁₂R₁₃)ᵣR₇, O(CR₁₂R₁₃)ₘNR₈R₉, NR₁₀C(O)(CR₁₂R₁₃)ᵣR₇, NR₁₀C(O)(CR₁₂R₁₃)ᵣOR₇, NR₁₀C(═NC)(CR₁₂R₁₃)ᵣR₇, NR₁₀CO(CR₁₂R₁₃)ᵣNR₈R₉, NR₁₀(CR₁₂R₁₃)ₘOR₇, NR₁₀(CR₁₂R₁₃)ᵣCO₂R₇, NR₁₀(CR₁₂R₁₃)ₘNR₈R₉, NR₁₀(CR₁₂R₁₃)ₙSO₂(CR₁₄R₁₅)_{q}R₇, CONR₁₀(CR₁₂R₁₃)ₙSO₂(CR₁₄R₁₅)_{q}R₇, SO₂NR₁₀(CR₁₂R₁₃)ₙCO(CR₁₄R₁₅)_{q}R₇, and SO₂NR₁₀(CR₁₂R₁₃)ₘOR₇ as well as pentafluorophenyl.

The terms “heterocycle”, “heterocyclic”, “heterocyclic group” or “heterocycle” refer to fully saturated or partially unsaturated cyclic groups (for example, 3 to 13 member monocyclic, 7 to 17 member bicyclic, or 10 to 20 member tricyclic ring systems, preferably containing a total of 3 to 10 ring atoms) which have at least one heteroatom in at least one carbon atom-containing ring. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system. The rings of multi-ring heterocycles may be either fused, bridged and/or joined through one or more spiro unions. Exemplary heterocyclic groups include

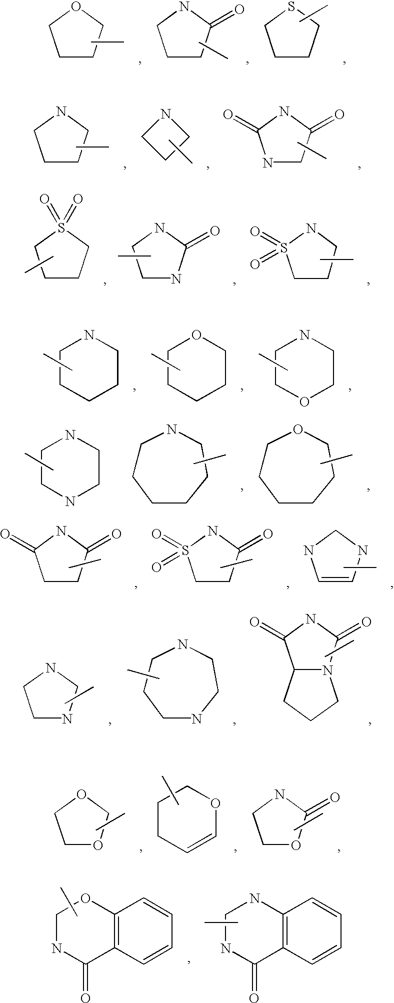

and the like.

The terms “substituted heterocycle” or “substituted heterocyclo” and the like refer to such heterocylo groups as defined above substituted with one or more groups listed in the definition of T¹, T² and T³, preferably selected from halogen, nitro, alkyl, substituted alkyl, alkenyl, cyano, cycloalkyl, substituted cycloalkyl, aryl, substituted aryl, heterocyclo, heteroaryl, oxo, OR₇, CO₂R₇, C(O)NR₈R₉, OC(O)R₇, OC(O)OR₇, OC(O)NR₈R₉, OCH₂CO₂R₇, C(O)R₇, NR₈R₉, NR₁OC(O)R₇, NR₁OC(O)OR₇, NR₁OC(O)C(O)OR₇, NR₁₀C(O)C(O)NR₈R₉, NR₁₀C(O)C(O)alkyl, NR₁₀C(NCN)OR₇, NR₁₀C(O)NR₈R₉, NR₁₀C(NCN)NR₈R₉, NR₁OC(NR₁₁)NR₈R₉, NR₁₀SO₂NR₈R₉, N₁₀SO₂R₇, SR₇, S(O)R₇, SO₂R₇, SO₃R₇, SO₂NR₈R₉, NHOR₇, NR₁₀NR₈R₉, N(COR₇)OR₁₀, N(CO₂R₇)OR₁₀, C(O)NR₁₀(CR₁₂R₁₃)ᵣR₇, CO(CR₁₂R₁₃)ₚO(CR₁₄R₁₅)_{q}CO₂R₇, CO(CR₁₂R₁₃)ᵣOR₇, CO(CR₁₂R₁₃)ₚO(CR₁₄R₁s)_{q}R₇, CO(CR₁₂R₁₃)ᵣNR₈R₉, OC(O)O(CR₁₂R₁₃)ₘNR₈R₉, OC(O)N(CR₁₂R₁₃)ᵣR₇, O(CR₁₂R₁₃)ₘNR₈R₉, NR₁OC(O)(CR₁₂R₁₃)ᵣR₇, NR₁OC(O)(CR₁₂R₁₃)ᵣOR₇, NR₁OC(═NC)(CR₁₂R₁₃)ᵣR₇, NR₁₀CO(CR₁₂R₁₃)ᵣNR₈R₉, NR₁₀(CR₁₂R₁₃)ₘOR₇, NR₁₀(CR₁₂R₁₃)ᵣCO₂R₇, NR₁₀(CR₁₂R₁₃)ₘNR₈R₉, NR₁₀(CR₁₂R₁₃)ₙSO₂(CR₁₄R₁₅)_{q}R₇, CONR₁₀(CR₁₂R₁₃)ₙSO₂(CR₁₄R₁₅)_{q}R₇, SO₂NR₁₀(CR₁₂R₁₃)ₙCO(CR₁₄R₁₅)_{q}R₇, and SO₂NR₁₀(CR₁₂R₁₃)ₘOR₇.

The term “heteroaryl” as used herein alone or as part of another group refers to a 5-6- or 7-membered aromatic rings containing from 1 to 4 nitrogen atoms and/or 1 or 2 oxygen or sulfur atoms provided that the ring contains at least 1 carbon atom and no more than 4 heteroatoms. The heteroaryl ring is linked through an available carbon or nitrogen atom. Also included within the definition of heteroaryl are such rings fused to a cycloalkyl, aryl, cycloheteroalkyl, or another heteroaryl ring. One, two, or three available carbon or nitrogen atoms in the heteroaryl ring can be optionally substituted with substituents listed in the description of T₁, T₂ and T₃. Also an available nitrogen or sulfur atom in the heteroaryl ring can be oxidized. Examples of heteroaryl rings include

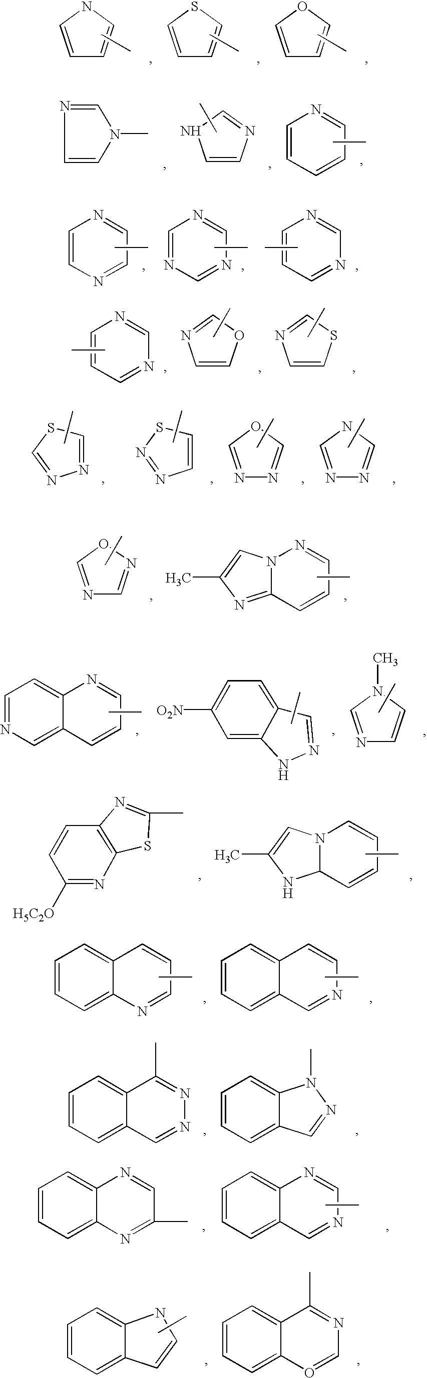

The term “substituted heteroaryl” refers to such heteroaryl groups as defined above substituted on any available atom with one or more groups listed in the definition of T¹, T² and T³, preferably selected from” refers to such heterocylo groups as defined .above substituted with one or more groups listed in the definition of T¹, T² and T³, preferably selected from halogen, nitro, alkyl, substituted alkyl, alkenyl, cyano, cycloalkyl, substituted cycloalkyl, aryl, substituted aryl, heterocyclo, heteroaryl, OR₇, CO₂R₇, C(O)NR₈R₉, OC(O)R₇, OC(O)OR₇, OC(O)NR₈R₉, OCH₂CO₂R₇, C(O)R₇, NR₈R₉, NR₁₀C(O)R₇, NR₁₀C(O)OR₇, NR₁₀C(O)C(O)OR₇, NR₁₀C(O)C(O)NR₈R₉, NR₁₀C(O)C(O)alkyl, NR₁₀C(NCN)OR₇, NR₁₀C(O)NR₈R₉, NR₁OC(NCN)NR₈R₉, NR₁OC(NR₁₁)NR₈R₉, NR₁₀SO₂NR₈R₉, NR₁₀SO₂R₇, SR₇, S(O)R₇, SO₂R₇, SO₃R₇, SO₂NR₈R₉, NHOR₇, NR₁₀NR₈R₉, N(COR₇)OR₁₀, N(CO₂R₇)OR₁₀, C(O)NR₁₀(CR₁₂R₁₃)ᵣR₇, CO(CR₁₂R₁₃)ₚO(CR₁₄R₁₅)_{q}CO₂R₇, CO(CR₁₂R₁₃)ᵣOR₇, CO(CR₁₂R₁₃)ₚO(CR₁₄R₁₅)_{q}R₇, CO(CR₁₂R₁₃)ᵣNR₈R₉, OC(O)O(CR₁₂R₁₃)ₘNR₈R₉, OC(O)N(CR₁₂R₁₃)ᵣR₇, O(CR₁₂R₁₃)ₘNR₈R₉, NR₁₀C(O)(CR₁₂R₁₃)ᵣR₇, NR₁₀C(O)(CR₁₂R₁₃)ᵣOR₇, NR₁OC(═NC)(CR₁₂R₁₃)ᵣR₇, NR₁₀CO(CR₁₂R₁₃)ᵣNR₈R₉, NR₁₀(CR₁₂R₁₃)ₘOR₇, NR₁₀(CR₁₂R₁₃)ᵣCO₂R₇, NR₁₀(CR₁₂R₁₃)ₘNR₈R₉, NR₁₀(CR₁₂R₁₃)ₙSO₂(CR₁₄R₁₅)_{q}R₇, CONR₁₀(CR₁₂R₁₃)ₙSO₂(CR₁₄R₁₅)_{q}R₇, SO₂NR₁₀(CR₁₂R₁₃)ₙCO(CR₁₄R₁₅)_{q}R₇, and SO₂NR₁₀(CR₁₂R₁₃)ₘOR₇.

R₇, R₁₀, and R₁₁, are independently selected from the group consisting of hydrogen, alkyl, substituted alkyl, alkenyl, alkynyl, cycloalkyl, substituted cycloalkyl, C(O)alkyl, C(O)substituted alkyl, C(O)cycloalkyl, C(O) substituted cycloalkyl, C(O)aryl, C(O)substituted aryl, C(O)Oalkyl, C(O)Osubstituted alkyl, C(O)heterocyclo, C(O)heteroaryl, aryl, substituted aryl, heterocyclo and heteroaryl.

R₈ and R₉ are independently selected from the group consisting of hydrogen, alkyl, substituted alkyl, cycloalkyl, substituted cycloalkyl, alkenyl, alkynyl, C(O)alkyl, C(O)substituted alkyl, C(O)cycloalkyl, C(O)substituted cycloalkyl, C(O)aryl, C(O)substituted aryl, C(O)Oalkyl, C(O)Osustituted alkyl, C(O)heterocyclo, C(O)heteroaryl, S(O)₂alkyl, S(O)₂substituted alkyl, S(O)₂cycloalkyl, S(O)₂substituted cycloalkyl, S(O)₂aryl, S(O)₂substituted aryl, S(O)₂heterocyclo, S(O)₂heteroaryl, aryl, substituted aryl, heterocyclo, and heteroaryl or R₈ and R₉ taken together with the nitrogen atom to which they are attached complete a heterocyclo or heteroaryl ring.

R₁₂ and R₁₄ are independently selected from hydrogen and alkyl or 1 to 4 carbons.

R₁₃ and R₁₅ are independently selected from hydrogen, alkyl of 1 to 4 carbons, and substituted alkyl or 1 to 4 carbons.

n is zero or an integer from 1 to 4.

m is an integer from 2 to 6.

p is an integer from 1 to 3.

q is zero or an integer from 1 to 3.

r is zero or an integer from 1 to 6.

T¹, T², and T³ are are each independently

(1) hydrogen or T⁶, where T⁶ is

(i) alkyl, (hydroxy)alkyl, (alkoxy)alkyl, alkenyl, alkynyl, cycloalkyl, (cycloalkyl)alkyl, cycloalkenyl, (cycloalkenyl)alkyl, aryl, (aryl)alkyl, heterocyclo, (heterocylco)alkyl, heteroaryl, or (heteroaryl)alkyl;

(ii) a group (i) which is itself substituted by one or more of the same or different groups (i); or

(iii) a group (i) or (ii) which is independently substituted by one or more (preferably 1 to 3) of the following groups (2) to (13) of the definition of T¹, T² and T³;

(2) —OH or —OT⁶,

(3) —SH or —ST⁶,

(4) —C(O)ₜH, —C(O)ₜT⁶, or —O—C(O)T⁶, where t is 1 or 2;

(5) —SO₃H, —S(O)ₜT⁶, or S(O)ₜN(T⁹)T⁶,

(6) halo,

(7) cyano,

(8) nitro,

(9) —T⁴—NT⁷T⁸,

(10) —T⁴—N(T⁹)—T⁵—NT⁷T^{8,}

(11) —T⁴—N(T¹⁰)—T⁵—T⁶,

(12) —T⁴—N(T¹⁰)—T⁵—H,

(13) oxo,

T4 and T⁵ are each independently

(1) a single bond,

(2) —T¹¹—S(O)ₜ—T¹²—,

(3) —T¹¹—C(O)—T¹²—,

(4) —T¹¹—C(S)—T¹²—,

(5) —T¹¹—O—T¹²—,

(6) —T¹¹—S—T¹²—,

(7) —T¹¹—O—C(O)—T¹²—,

(8) —T¹¹—C(O)—O—T¹²—,

(9) —T¹¹—C(═NT^{9a})—T¹²—, or

(10) —T¹¹—C(O)—C(O)—T¹²—

T⁷, T⁸, T^{9, T}^{9a} and T¹⁰

(1) are each independently hydrogen or a group provided in the definition of T⁶, or

(2) T⁷ and T⁸ may together be alkylene or alkenylene, completing a 3- to 8-membered saturated or unsaturated ring together with the atoms to which they are attached, which ring is unsubstituted or substituted with one or more groups listed in the description of T¹, T² and T³, or

(3) T⁷ or T⁸, together with T⁹, may be alkylene or alkenylene completing a 3- to 8-membered saturated or unsaturated ring together with the nitrogen atoms to which they are attached, which ring is unsubstituted or substituted with one or more groups listed in the description of T¹, T² and T³, or

(4) T⁷ and T⁸ or T⁹ and T¹⁰ together with the nitrogen atom to which they are attached may combine to form a group —N═CT¹³T¹⁴ where T¹³ and T¹⁴ are each independently H or a group provided in the definition of T⁶; and

T¹¹ and T¹² are each independently

(1) a single bond,

(2) alkylene,

(3) alkenylene, or

(4) alkynylene.

“T cell-mediated diseases” refers to any disorder or disease state in which modulation of the activity of T cells is implicated in a process which results in either a pathophysiological state or a process where the normal function of T cells is intended to be suppressed for therapeutic benefit. Examples of T cell mediated disorders include transplant rejection, graph verses host disease, and autoimmune disorders, such as rheumatoid arthritis, multiple sclerosis, juvenile diabetes, asthma, and inflammatory bowel disease, T-cell mediated hypersensitivity diseases, ischemic or reperfusion injury, and T-cell proliferative disorders.

PDE7 inhibitors in accordance with the present invention are employed, typically in the form of a pharmaceutical composition including a pharmaceutically acceptable carrier for the treatment of T-cell mediated disease. The compounds employed for this purpose are typically administered in an amount from about 0.01 to 100 mg/kg/day.

The pharmaceutical compositions comprising at least one PDE7 inhibitor may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

The PDE7 inhibitors may be administered by any suitable means, for example, orally, such as in the form of tablets, capsules, granules or powders; sublingually; buccally; parenterally, such as by subcutaneous, intravenous, intramuscular, or intrasternal injection or infusion techniques (e.g., as sterile injectable aqueous or non-aqueous solutions or suspensions); nasally such as by inhalation spray; topically, such as in the form of a cream or ointment; or rectally such as in the form of suppositories; in dosage unit formulations containing non-toxic, pharmaceutically acceptable vehicles or diluents. The present compounds may, for example, be administered in a form suitable for immediate release or extended release. Inmediate release or extended release may be achieved by the use of suitable pharmaceutical compositions comprising the present compounds, or, particularly in the case of extended release, by the use of devices such as subcutaneous implants or osmotic pumps. The present compounds may also be administered in the form of liposomes.

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants such as those known in the art. The present compounds may also be delivered through the oral cavity by sublingual and/or buccal administration. Molded tablets, compressed tablets or freeze-dried tablets are exemplary forms which may be used. Exemplary compositions include those formulating the present compound(s) with fast dissolving diluents such as mannitol, lactose, sucrose and/or cyclodextrins. Also included in such formulations may be high molecular weight excipients such as celluloses (avicel) or polyethylene glycols (PEG). Such formulations may also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g., Carbopol 934). Lubricants, glidants, flavors, coloring agents and stabilizers may also be added for ease of fabrication and use.

Exemplary compositions for nasal aerosol or inhalation administration include solutions in saline which may contain, for example, benzyl alcohol or other suitable preservatives, absorption promoters to enhance bioavailability, and/or other solubilizing or dispersing agents such as those known in the art.

Exemplary compositions for parenteral administration include injectable solutions or suspensions which may contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid.

Exemplary compositions for rectal administration include suppositories which may contain, for example, a suitable non-irritating excipient, such as cocoa butter, synthetic glyceride esters or polyethylene glycols, which are solid at ordinary temperatures, but liquefy and/or dissolve in the rectal cavity to release the drug.

Exemplary compositions for topical administration include a topical carrier such as Plastibase (mineral oil gelled with polyethylene).

The effective amount of a compound employed in the present invention may be determined by one of ordinary skill in the art, and includes exemplary dosage amounts for an adult human of from about 0.01 to 100 mg/kg of body weight of active compound per day, which may be administered in a single dose or in the form of individual divided doses, such as from 1 to 4 times per day. It will be understood that the specific dose level and frequency of dosage for any particular subject may be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats and the like, subject to inflammatory, immunological, or respiratory cell-associated disorders.

PDE7 inhibitors for use in the treatment of various T-cell mediated diseases are those covered by Formula I.

Compounds of Formula I include salts, prodrugs and solvates. The term “salt(s)”, as employed herein, denotes acidic and/or basic salts formed with inorganic and/or organic acids and bases. Zwitterions (internal or inner salts) are included within the term “salt(s)” as used herein (and may be formed, for example, where the R substituents comprise an acid moiety such as a carboxyl group). Also included herein are quaternary ammonium salts such as alkylammonium salts. Pharmaceutically acceptable (i.e., non-toxic, physiologically acceptable) salts are preferred, although other salts are useful, for example, in isolation or purification steps which may be employed during preparation. Salts of the compounds of the formula I may be formed, for example, by reacting a compound I with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates, undecanoates, and the like.

Exemplary basic salts (formed, for example, where the R substituents comprise an acidic moiety such as a carboxyl group) include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines, N-methyl-D-glucamines, N-methyl-D-glucamides, t-butyl amines, and salts with amino acids such as arginine, lysine and the like. The basic nitrogen-containing groups may be quatemized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

Prodrugs and solvates of the compounds of the invention are also contemplated herein. The term “prod rug”, as employed herein, denotes a compound which, upon administration to a subject, undergoes chemical conversion by metabolic or chemical processes to yield a compound of the Formula I, or a salt and/or solvate thereof. Solvates of the compounds of Formula I are preferably hydrates.

All stereoisomers of the present compounds, such as those which may exist due to asymmetric carbons on the R substituents of the compound of the formula I, including enantiomeric and diastereomeric forms, are contemplated within the scope of this invention. Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations.

The compounds of Formula I are typically employed as part of a pharmaceutical composition including a pharmaceutically acceptable carrier for the treatment of respiratory and non-respiratory diseases. The compounds employed for this purpose are typically administered in an amount of from about 0.01 to 100 mg/kg/day. The compounds of Formula I are especially effective in inhibiting the PDE7 enzyme. Additionally a subset of compounds are also effective at inhibiting PDE4.

The pharmaceutical composition comprising at least one compound of Formula I may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

The compounds of Formula I may be administered by any suitable means, for example, orally, such as in the form of tablets, capsules, granules or powders; sublingually; bucally; parenterally, such as by subcutaneous, intravenous, intramuscular, or intrasternal injection or infusion techniques (e.g., as sterile injectable aqueous or non-aqueous solutions or suspensions); nasally such as by inhalation spray; topically, such as in the form of a cream or ointment; or rectally such as in the form of suppositories; in dosage unit formulations containing non-toxic, pharmaceutically acceptable vehicles or diluents. The present compounds may be based for immediate release or extended release by the use of suitable pharmaceutical compositions comprising the present compounds, or, particularly in the case of extended release, by the use of devices such as subcutaneous implants or osmotic pumps. The present compounds may also be administered liposomally.

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants such as those known in the art. The present compounds may also be delivered through the oral cavity by sublingual and/or buccal administration. Molded tablets, compressed tablets or freeze-dried tablets are exemplary forms which may be used. Exemplary compositions include those formulating the present compound(s) with fast dissolving diluents such as mannitol, lactose, sucrose and/or cyclodextrins. Also included in such formulations may be high molecular weight excipients such as celluloses (avicel) or polyethylene glycols (PEG). Such formulations may also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g., Carbopol 934). Lubricants, glidants, flavors, coloring agents and stabilizers may also be added for ease of fabrication and use.

Exemplary compositions for nasal aerosol or inhalation administration include solutions in saline which may contain, for example, benzyl alcohol or other suitable preservatives, absorption promoters to enhance bioavailability, and/or other solubilizing or dispersing agents such as those known in the art.

Exemplary compositions for parenteral administration include injectable solutions or suspensions which may contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid.

Exemplary compositions for rectal administration include suppositories which may contain, for example, a suitable non-irritating excipient, such as cocoa butter, synthetic glyceride esters or polyethylene glycols, which are solid at ordinary temperatures, but liquefy and/or dissolve in the rectal cavity to release the drug.

Exemplary compositions for topical administration include a topical carrier such as Plastibase (mineral oil gelled with polyethylene).

The effective amount of a compound of the present invention may be determined by one of ordinary skill in the art, and includes exemplary dosage amounts for an adult human from about 0.01 to 100 mg/kg of body weight of active compound per day, which may be administered in a single dose or in the form of individual divided doses, such as from 1 to 4 times per day. It will be understood that the specific dose level and frequency of dosage for any particular subject may be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats and the like, subject to leukocyte activation or respiratory cell-associated disorders.

### Methods of Preparation

Compounds of Formula I may be prepared by reference to the methods illustrated in the following Schemes A through C. As shown therein the end product is a compound having the same structural formula as Formula I. It will be understood that any compound of Formula I may be produced by Scheme A and B by the suitable selection of appropriate substitution. Schemes C shows the preparation of amides from compounds of Formula I derived from Schemes A and B. Solvents, temperatures, pressures, and other reaction conditions may readily be selected by one of ordinary skill in the art. All documents cited are incorporated herein by reference in their entirety. Starting materials are commercially available or readily prepared by one of ordinary skill in the art. Constituents of compounds are as defined herein or elsewhere in the specification.

Compounds within the scope of the present invention may be prepared by several methods, including condensation of a cyclic beta-keto esters with an appropriately substituted guanidine to provide compounds of formula I as illustrated in synthetic Scheme A1 In this case guanidine A1 is heated with a cyclic beta-keto ester A2 produce intermediate A3 reaction with phosphorous oxychloride provides intermediate A4. Reaction with reagent A5, which may be an amine, an alcohol, a thiol or a sulfonamide on the presence of a suitable base to provide compound A6 which is a compound of formula IIa, IIb, IIIa, IIIb, IIIc, or IV

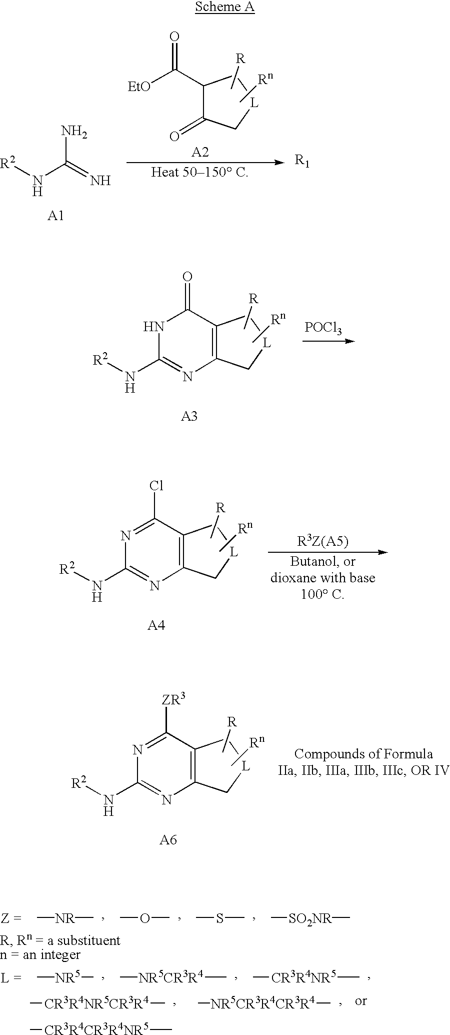

Cyclic beta-keto esters of structure A2, are either commercially available, or readily prepared by one of the methods outlined in Schemes B1, B2, B3, or B4. In scheme B I an amine B 1.1 is reacted with dialkylacrylate B1.2 to provide the di-addition product B1.3. Reaction with a base such as sodium alkoxide results in a Dieckmann cyclization to produce keto ester B1.4.

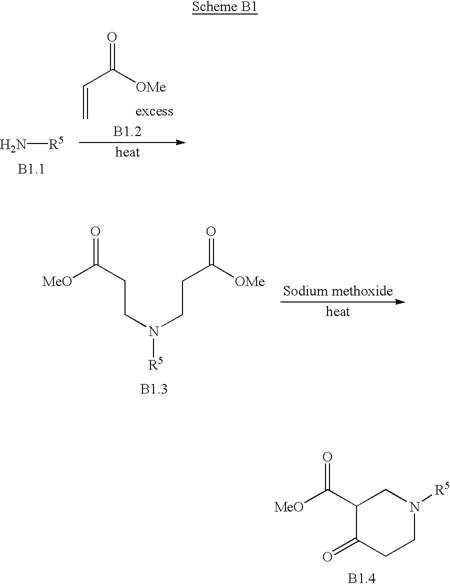

Regioisomeric six membered beta-ketoesters of structure B2.7 are either commercially available of prepared by methods which have been reported in the literature (for example Prill, E. et. al. J. Am. Chem. Soc. (1933) 557 1233.), and are outlined in Scheme B2. Thus an N-alkylated amino acid B2.1 which is either commercially available or readily prepared according to a number of methods reported in the literature is reacted with ethyl bromocrotonate B2.2 to yield intermediate B2.3 which undergoes double bond reduction to yield B2.4 which is reacted under standard Dieckmann cyclization conditions to yield intermediate B2.5. If a convenient amine protecting group, such as benzyl group, has been utilized removal under a variety of condition such as hydrogenation or reaction with a chloroformate reagent would provide the free amine. Regiospecific alkylation of the amine B2.6 has been reported in the literature (DaSilva-Goes, A., et. al. Tetrahedron Lett. (1998) 1339-40.) to provide compounds B2.7.

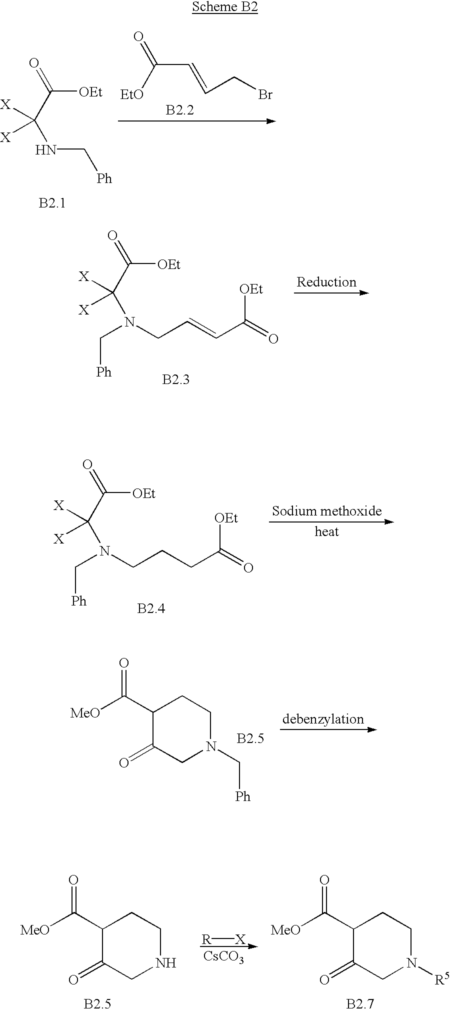

The synthesis of seven member cyclic beta-keto esters of structure B3.2, B3.4 and B3.5 are described in Scheme B3. B3.2 can be prepared from piperidones B2.1, which are either commercially available or can be prepared by a number of methods, including decarboxylation of B 1.4 with reagents such as sodium bromide at elevated temperature. Treatment of the piperidone B2.1, with ethyl diazoacetate and boron trifluoride etherate at reduced temperature provide the ring expanded intermediate B2.2, useful for the preparation of compounds of formula IIIa. Non-symmetrical piperidones B3.3 are either commercially available and have been reported in the literature, or they may also be prepared by decarboxylation of intermediate B2.7 (Krosgsgaard-Larsen, P., and Hjeds, H., Acta Chem. Scand. Ser. B. (1976) 884-88.). Piperidones B3.3 react with ethyl diazoacetate to produce a separable mixture of seven membered ring regioisomers. Selection of the desired regioisomer and reaction as depicted in scheme A would be useful for the production of compounds of formula IIIb or IIIc.

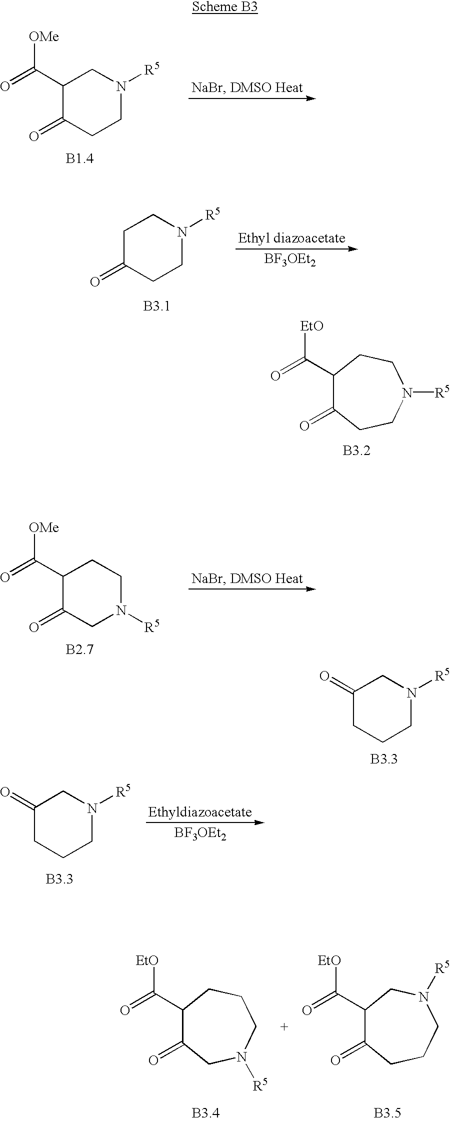

Five-membered cyclic beta-ketoesters are either commercially available or may be prepared from intermediates B2.1 followed by Michael addition to an appropriate acrylate B4.1 to produce intermediate B4.2. Condensation in the presence of titanium tetrachloride has been reported in the literature (Deshmukh, M. N., et. al. Synth. Comm. (1996) 26(9) 1657.) to produce compounds of type B4.3. Removal of a protecting group, such as a benzyl group could provide a diversity of compounds.

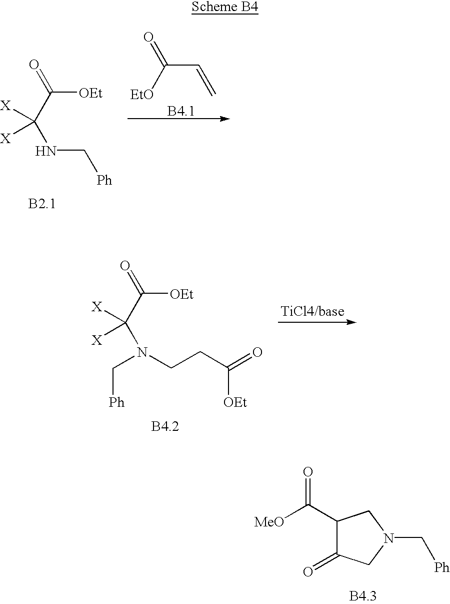

Scheme C outlines the conversion of esters of Formula I to amides of Formula I. Hydrolysis of the ester of compound C1.1 under basic conditions such as sodium hydroxide affords the acid C2. Alternatively judicious choice of protecting groups such as a tert-butyl group as in compound C1.2 may be readily removed by treatment with trifluoroacetic acid to produce acid C2. A second alternative is to use a benzyl protecting group, as in compound C1.2 which may be removed by reaction with hydrogen in the presence of a suitable catalyst such as palladium on carbon under elevated pressure. Coupling of acid C2 under standard amide bond coupling techniques (DIC/HOAt) with the appropriate amine C3 gives the desired amide C4.

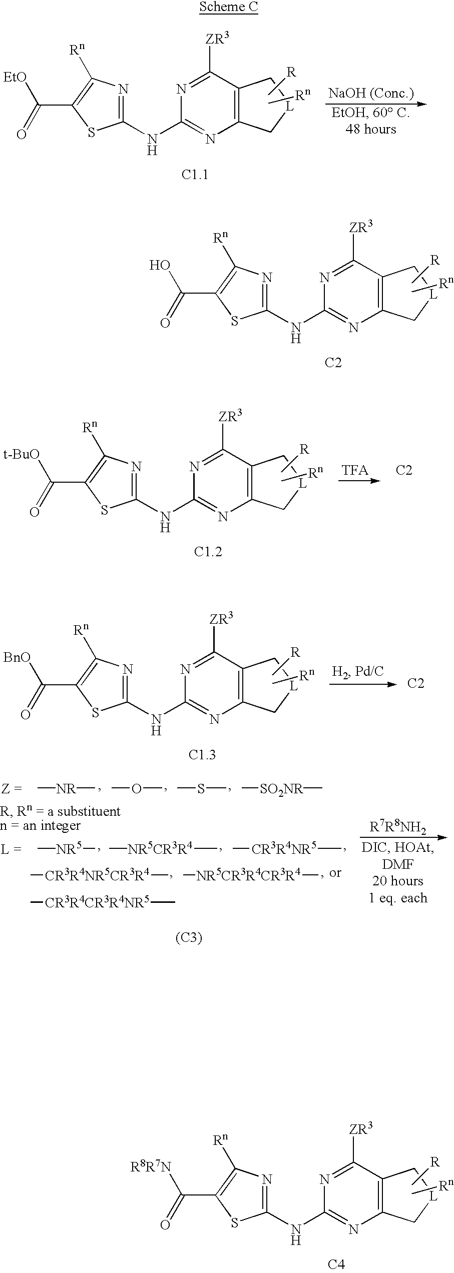

Appropriately substituted guanidines referred to in scheme A, are either commercially available or readily prepared by a number of methods known to one skilled in the art of organic chemistry. As depicted in scheme D1, amines D1.1 may be reacted with a number of reagents such as the commercially available 2-3,5-dimethylpyrazole-1-carboxamidine nitrate D1.2 to provide the desired guanidine D1.3

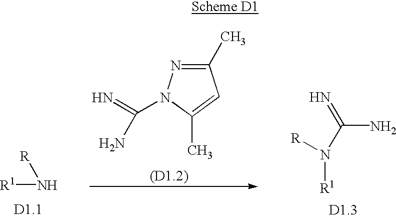

In some instances it is more convenient to prepare the intermediate guanidines XIX as illustrated in Scheme D2. alpha-Haloketone D2.1 is reacted with thiobiuret, D2.2, to provide the guanidine salt D2.3, which is liberated by treatment with a basic resin, or sodium hydroxide, sodium methoxide, or an amine base to provide intermediate D2.4, which can be further elaborated as described in Scheme A to provide compounds of formula I.

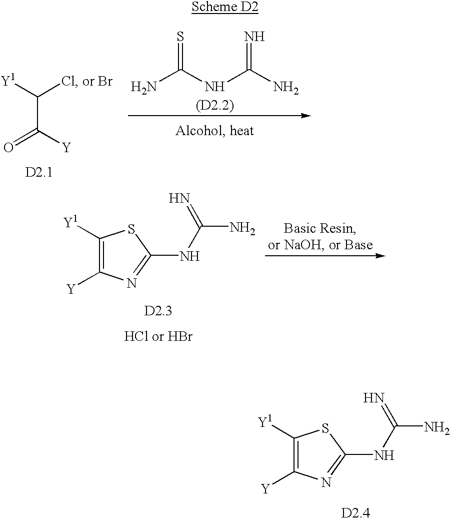

### Utility

Selective PDE7 inhibitors or dual PDE7-PDE4 inhibitors including compounds of formulas I, are useful in the treatment (including prevention, partial alleviation or cure) of leukocyte activation-associated disorders, which include (but are not limited to) disorders such as: transplant rejection (such as organ transplant, acute transplant, xenotransplant or heterograft or homograft such as is employed in burn treatment); protection from ischemic or reperfusion injury such as ischemic or reperfusion injury incurred during organ transplantation, myocardial infarction, stroke or other causes; transplantation tolerance induction; arthritis (such as rheumatoid arthritis, psoriatic arthritis or osteoarthritis); multiple sclerosis; respiratory and pulmonary diseases including but not limited to asthma, exercise induced asthma, chronic obstructive pulmonary disease (COPD), emphysema, bronchitis, and acute respiratory distress syndrome (ARDS); inflammatory bowel disease, including ulcerative colitis and Crohn's disease; lupus (systemic lupus erythematosis); graft vs. host disease; T-cell mediated hypersensitivity diseases, including contact hypersensitivity, delayed-type hypersensitivity, and gluten-sensitive enteropathy (Celiac disease); psoriasis; contact dermatitis (including that due to poison ivy); Hashimoto's thyroiditis; Sjogren's syndrome; Autoimmune Hyperthyroidism, such as Graves' Disease; Addison's disease (autoimmune disease of the adrenal glands); Autoimmune polyglandular disease (also known as autoimmune polyglandular syndrome); autoimmune alopecia; pernicious anemia; vitiligo; autoimmune hypopituatarism; Guillain-Barre syndrome; other autoimmune diseases; glomerulonephritis; serum sickness; uticaria; allergic diseases such as respiratory allergies (e.g., asthma, hayfever, allergic rhinitis) or skin allergies; scleracierma; mycosis fungoides; acute inflammatory and respiratory responses (such as acute respiratory distress syndrome and ishchemia/reperfusion injury); dermatomyositis; alopecia areata; chronic actinic dermatitis; eczema; Behcet's disease; Pustulosis palmoplanteris; Pyoderma gangrenum; Sezary's syndrome; atopic dermatitis; systemic schlerosis; and morphea.

The term “leukocyte activation-associated disorder” or “leukocyte activation-mediated disorder” as used herein includes each of the above referenced diseases or disorders. The compounds of the present invention are useful for treating the aforementioned exemplary disorders irrespective of their etiology.

Those present compounds which are dual PDE7/4 inhibitors may be more effective than either a selective PDE4 inhibitor or a selective PDE7 inhibitor in the above mentioned disease states, as a result of either additive or synergistic activity resulting from the combined inhibition of PDE7 and PDE4.

The present invention thus provides methods for the treatment of disorders as discussed above comprising the step of administering to a subject in need thereof of at least one selective PDE7 inhibitor or at least one dual PDE7-PDE4 inhibitor for the treatment of leukocyte activation-associated or leukocyte-activation mediated disease. Other therapeutic agents such as those described below may be employed with the compounds of the present invention. In the methods of the present invention, such other therapeutic agent(s) may be administered prior to, simultaneously with or following the administration of the compound(s) of the present invention.

The methods of treating diseases which would benefit from the inhibition of PDE7 or the inhibition of both PDE7-PDE4 by a dual agent may comprise administering compounds of Formula (I) alone or in combination with each other and/or other suitable therapeutic agents useful in treating such conditions such as: immunosuppressants such as, cyclosporins (e.g., cyclosporin A), anti-IL-1 agents, such as Anakinra, the IL-i receptor antagonist, CTLA4-Ig. antibodies such as anti-ICAM-3, anti-IL-2 receptor (Anti-Tac), anti-CD45RB, anti-CD2, anti-CD3, anti-CD4, anti-CD80, anti-CD86, monoclonal antibody OKT3, agents blocking the interaction between CD40 and CD154, such as antibodies specific for CD40 and/or CD154 (i.e., CD40L), fusion proteins constructed from CD40 and CD154 (CD401 g and CD8-CD154), interferon beta, interferon gamma, methotrexate, FK506 (tacrolimus, Prograf), rapamycin (sirolimus or Rapamune)mycophenolate mofetil, leflunomide (Arava), azathioprine and cyclophosphamide, inhibitors, such as nuclear translocation inhibitors, of NF-kappa B function, such as deoxyspergualin (DSG), non-steroidal antiinflammatory drugs (NSAIDs) such as ibuprofen, cyclooxygenase-2 (COX-2) inhibitors such as celecoxib (Celebrex) and rofecoxib (Vioxx), or derivatives thereof, steroids such as prednisone or dexamethasone, gold compounds TNF-α inhibitors such as tenidap, anti-TNF antibodies or soluble TNF receptor such as etanercept (Enbrel), inhibitors of p-38 kinase such as BIRB-796, RO-3201195, VX-850, and VX-750, beta-2 agonists such as albuterol, levalbuterol (Xopenex), and salmeterol (Serevent), inhibitors of leukotriene synthesis such as inontelikast (Singulair) and zariflukast (Accolate), and anticholinelegic agents such as ipratropium bromide (Atrovent), PDE4 inhibitors such as Arofyline, Cilomilast, Roflumilast, C-11294A, CDC-801, BAY-19-8004, Cipamfylline, SCH351591, YM-976, PD-189659, Mesiopram, Pumafentrine, CDC-998, IC-485, and KW-4490, PDE7 inhibitors such as IC242, (Lee, et. al. PDE7A is expressed in human B-lymphocytes and is up-regulated by elevation of intracellular cAMP. Cell Signalling, 14, 277-284, (2002)) and also include compounds disclosed in the following patent documents: WO 0068230, WO 0129049, WO 0132618, WO 0134601, WO 0136425, WO 0174786, WO 0198274, WO 0228847, U.S. Provisional Application Serial No. 60/287,964, and U.S. Provisional Application Serial No. 60/355,141anti-cytokines such as anti-IL-1 mAb or IL-1 receptor agonist, anti-IL-4 or IL-4 receptor fusion proteins and PTK inhibitors such as those disclosed in the following U.S. Patents and Applications, incorporated herein by reference in their entirety: U.S. Patent No. 6,235,740, U.S. Pat. No. 6,239,133, U.S. Application Serial No. 60/065,042, filed Nov. 10, 1997 (Attorney Docket No. QA207*), U.S. application Ser. No. 09/173,413, filed Oct. 15, 1998 (Attorney Docket No. QA 207a), and U.S. Pat. No. 5,990,109.

See the following documents and references cited therein: Hollenbaugh, D., Douthwright, J., McDonald, V., and Aruffo, A., “Cleavable CD401 g fusion proteins and the binding to sgp39”, J. Immunol. Methods (Netherlands), 188(1), p. 1-7 (Dec 15, 1995); Hollenbaugh, D., Grosmaire, L. S., Kullas, C. D., Chalupny, N. J., Braesch-Andersen, S., Noelle, R. J., Stamenkovic, I., Ledbetter, J. A., and Aruffo, A., “The human T cell antigen gp39, a member of the TNF gene family, is a ligand for the CD40 receptor: expression of a soluble form of gp39 with B cell co-stimulatory activity”, EMBO J. (England), 11(12), p 4313-4321 (Dec 1992); and Moreland, L. W. et al., “Treatment of rheumatoid arthritis with a recombinant human tumor necrosis factor receptor (p75)-Fc fusion protein, New England J. of Medicine, 337(3), p. 141-147 (1997).

Compounds present invention (especially selective PDE 7 inhibitors) may also be employed in combination with PDE 4 inhibitors. Examples of selective PDE4 inhibitors currently in development, which can be used in combination with compounds of the present invention include Arofyline, Cilomilast, Roflumilast, C-11294A, CDC-801, BAY-19-8004, Cipamfylline, SCH351591, YM-976, PD-189659, Mesiopram, Pumafentrine, CDC-998, IC-485, and KW-4490.

The above other therapeutic agents, when employed in combination with the compounds of the present invention, may be used, for example, in those amounts indicated in the Physicians' Desk Reference (PDR) or as otherwise determined by one of ordinary skill in the art.

Use of the compounds of the present invention as encompassed by formula I in treating leukocyte activation-associated disorders is exemplified by, but is not limited to, treating a range of disorders such as: transplant (such as organ transplant, acute transplant, xenotransplant or heterograft or homograft (such as is employed in burn treatment)) rejection; protection from ischemic or reperfusion injury such as ischemic or reperfusion injury incurred during organ transplantation, myocardial infarction, stroke or other causes; transplantation tolerance induction; arthritis (such as rheumatoid arthritis, psoriatic arthritis or osteoarthritis); multiple sclerosis; respiratory and pulmonary diseases including but not limited to asthma, exercise induced asthma, chronic obstructive pulmonary disease (COPD), emphysema, bronchitis, and acute respiratory distress syndrome (ARDS); inflammatory bowel disease, including ulcerative colitis and Crohn's disease; lupus (systemic lupus erythematosis); graft vs. host disease; T-cell mediated hypersensitivity diseases, including contact hypersensitivity, delayed-type hypersensitivity, and gluten-sensitive enteropathy (Celiac disease); psoriasis; contact dermatitis (including that due to poison ivy); Hashimoto's thyroiditis; Sjogren's syndrome; Autoimmune Hyperthyroidism, such as Graves' Disease; Addison's disease ,(autoimmune disease of the adrenal glands); Autoimmune polyglandular disease (also known as autoimmune polyglandular syndrome); autoimmune alopecia; pernicious anemia; vitiligo; autoimmune hypopituatarism; Guillain-Barre syndrome; other autoimmune diseases; glomerulonephritis; serum sickness; uticaria; allergic diseases such as respiratory allergies (asthma, hayfever, allergic rhinitis) or skin allergies; scleracierma; mycosis fungoides; acute inflammatory and respiratory responses (such as acute respiratory distress syndrome and ishchemia/reperfusion injury); dermatomyositis; alopecia areata; chronic actinic dermatitis; eczema; Behcet's disease; Pustulosis palmoplanteris; Pyoderma gangrenum; Sezary's syndrome; atopic dermatitis; systemic schlerosis; and morphea.

The combined activity of the present compounds towards T-cells and other PDE7-expressing cells may be of value in the treatment of any of the aforementioned disorders. Additionally those present compounds which are dual PDE4/7 inhibitors may be more effective than either a selective PDE4 inhibitor or a selective PDE7 inhibitor in the above mentioned disease states.

In a particular embodiment, the compounds of the present invention are useful for the treatment of the aforementioned exemplary disorders irrespective of their etiology, for example, for the treatment of transplant rejection, rheumatoid arthritis, multiple sclerosis, chronic obstructive pulmonary disease, inflammatory bowel disease, lupus, graft v. host disease, T-cell mediated hypersensitivity disease, psoriasis, Hashimoto's thyroiditis, Guillain-Barre syndrome, cancer, contact dermatitis, allergic disease such as allergic rhinitis, asthma, ischemic or reperfusion injury, respiratory diseases such as asthma, COPD and bronchitis or atopic dermatitis whether or not associated with leukocyte activation.

PDE-Containing Cell Lysates

Hut78 cells were grown in 20% FCS in Iscoves Modified Dulbecco's Medium (Gibco BRL-Life Technologies, Grand Island, N.Y.) with antibiotics. Cells were centrifuged and resuspended in four volumes of [40 mM Tris (pH 7.5)/50 PM EDTA/200 uM PMSF with a cocktail of Protease inhibitors (Boehringher Mannheim, Indianapolis, Ind.)] at 4C. Cells were homogenized using a Dounce homogenizer, and the lysate was centrifuged for 30 min at 15,000× g. Glycerol was added to a final volume of 50% for storage at −20C.

SPA Assay

Inhibition of PDE activity in Hut78 cell lysate was determined using an SPA specific for cAMP (Amersham Pharmacia Biotech, Buckinghamshire, UK) according to the manufacturers instructions with minor modifications. Enzyme assays were performed at room temperature in the presence of 50 mM Tris HCi, pH 7.5, containing 8.3 mM MgCl₂, 1.7 mM EGTA and 0.3 mg/mL BSA. Each assay was performed in a 100 μL reaction volume in 96 well microtitre plates containing the above buffer, 0.3 ul of Hut78 cell lysate treated with 2 mM Zardaverine to inhibit PDE3 and PDE4, 0.05 uCi of [5′,8-₃H] Adenosine 3′,5′-cyclic phosphate as an ammonium salt for 20 min. The reaction was terminated by the addition of 50 μl PDE SPA beads (1 mg) suspended in 18 mM zinc sulphate with 10 mM cold cAMP (Sigma, St. Louis Mo.). The reaction mix was allowed to settle for 20 minutes before counting in a Top Count-NXT scintillation counter (Packard BioScience, Meriden, Conn.).

T Cell Proliferation Assay

Peripheral blood mononuclear cells (PBMC) were isolated from whole blood by density gradient centrifugation over Lymphoprep, 1.077. Cells were plated into 96 well U-bottom plates at 2.5x10₅ cells/well in 10% FBS RPMI 1640 (Life Technologies/Gibco-BRL) containing 10 ug/ml anti-CD3 (G19-4, Bristol-Myers Squibb P.R.I., Princeton, N.J.) and lug/ml anti-CD28 (9.3, Bristol-Myers Squibb P.R.I.) in the presence and absence of inhibitors. DMSO (used as a solvent for inhibitors) was added to the medium at 0.1% final concentration. The total volume per well was 200 μL. Cells were incubated at 37C. 5% CO₂ for 3 days, at which time 0.5μCi of ³H-thymidine was added to each well. Six hours following the addition of ³H-thmidine, the plates were harvested onto filter plates, 30 μl EcoLite scintillant (ICN, Costa Mesa, Calif.) was added per well, and plates read on a Top Count-NXT scintillation counter.

TNFα Secretion Assay

The ability of compounds to inhibit the production and secretion of TNFα from leukocytes was performed using either PBMC (obtained as described above) or the THP-1 cell line as a source of monocytes. Compounds were diluted in RPMI 1640 supplemented with 10% FBS and DMSO at a final concentration of 0.2%. Cells (2×10⁵/well in U-bottom 96 well plates) were pre-incubated with compounds for 30 min at 37 C prior to addition of lipopolysaccharide (LPS) at a final concentration of 6.25 ng/ml in a total volume of 200 μL. After 4 h at 37C, 50 μL of supernatant was carefully aspirated for detection of soluble TNFX. Soluble TNFα was detected by ELISA developed by R&D Systems (Minneapolis, Minn.) according to the manufacturers instructions.

### EXAMPLES

The following examples illustrate preferred embodiments of the present invention and do not limit the scope of the present invention which is defined in the claims. Abbreviations employed in the Examples are defined below. Compounds of the Examples are identified by the example and step in which they are prepared (e.g., “A1.1” denotes the title compound of step 1 of Example A1), or by the example only where the compound is the title compound of the example (for example, “A2” denotes the title compound of Example A2).

Abbreviations

| | |
|---|---|
| Ac | Acetyl |
| AcOH | Acetic acid |
| aq. | Aqueous |
| CDI | Carbonyldiimidazole |
| Bn | Benzyl |
| Bu | Butyl |
| Boc | tert-butoxycarbonyl |
| DMAP | Dimethylaminopyridine |
| DMA | N,N-Dimethylacetamide |
| DMF | dimethylformamide |
| DMSO | Dimethylsulfoxide |
| EDC | 1-(3-Dimethylaminopropyl)-3-ethylcarbodiimide |
| | hydrochloride |
| EtOAc | Ethyl acetate |
| Et | Ethyl |
| EtOH | Ethanol |
| H | Hydrogen |
| h | Hours |
| i | iso |
| HPLC | High pressure liquid chromatography |
| HOAc | Acetic acid |
| Lawesson's Reagent | [2,4-bis(4-methoxyphenyl)-1,3-dithia-2,4- |
| | diphosphetane-2-4-disufide |
| LC | liquid chromatography |
| Me | Methyl |
| MeOH | Methanol |
| min. | Minutes |
| M⁺ | (M + H)⁺ |
| M⁺¹ | (M + H)⁺ |
| MS | Mass spectrometry |
| n | normal |
| Pd/C | Palladium on carbon |
| Ph | Phenyl |
| Pr | Propyl |
| Ret Time | Retention time |
| rt or RT | Room temperature |
| sat. | Saturated |
| S-Tol-BINAP | (S)-(−)-2,2′-Bis(di-p-tolylphosphino)-1,1′-binapthyl |
| t | tert |
| TFA | Trifluoroacetic acid |
| THF | Tetrahydrofuran |
| YMC | YMC Inc, Wilmington, NC 28403 |

HPLC conditions used to determine retention times; 4 min gradient O-100% B in A(A; 0.1% TFA in 90/10 water/methanol; B; 0.1% TFA in 10/90 water/methanol) using a YMC turbopack column at with a detection wavelength of 220 nanometeres or 254 nanometers.

### Example A1

### ′2-[[4-[[[4-(Methylsulfonyl)phenyl]methyl]amino]-5,6,7,8-tetrahydro-6-methylpyridor[4,3-d]pyrimidin-2-yl]amino]-4-methyl-5-thiazolecarboxylic Acid Ethyl Ester

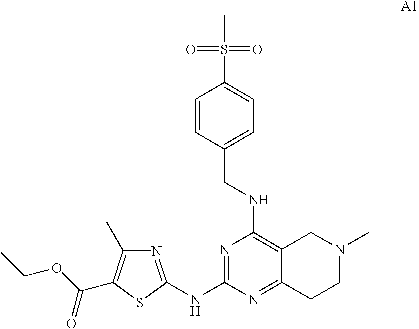

A1.1: N-(3-Methoxy-3-oxopropyl)-N-methyl-β-alanine Methyl Ester

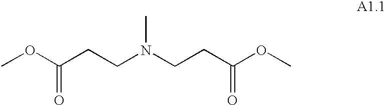

A solution of methyl acrylate (3.79 g, 44 mmol) and methyl amine (2M in methanol, 10 ml, 20 mmol)was heated to 100° C. in a sealed pressure tube for 2 days. The reaction mixture was concentrated to give a crude product which was purified on silica gel column with dichloromethane/methanol (50/1). The fractions which contained the product was concentrated and dried over vacuum pump to yield A1.1 (3.96 g, 86%). ¹H—NMR CDCl₃) δ:3.70 (6H, s), 2.74 (4H, t, J=7 Hz), 2.50 (41, t, J=7 Hz), 2.27( 3H, s).

A1.2: 1-Methyl-4-oxo-3-piperidinecarboxylic Acid Methyl Ester

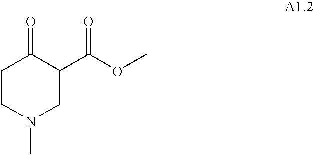

To a solution of sodium methoxide (25% in methanol, 4.74 ml, 20 mmol) in toluene (40 ml) at 110° C. was added A1.1 (2.0 g, 9.84 mmol). The reaction mixture was refluxed for 1 hr and then it was cooled down to room temperature. The reaction mixture was concentrated to give a crude product which was purified on silica gel column with dichloromethane/methanol (20/1). The fractions which contained the product was concentrated and dried over vacuum pump to yield the desired product (1.61 g, 96% ). ¹H—NMR (CD₃OD) δ: 3.50 (3H, s), 3.25 (1H, m), 3.09 (1H, m), 2.60-2.70 (1H, m), 2.44-2.51 (1H, m), 2.14-2.34 (5H, m). HPLC: 96%, ret. time=0.18 min., LC/MS (M+H)⁺=172.

A1.3: 2-[(Aminoiminomethyl)amino]-4-methyl-5-thiazolecarboxylic Acid Ethyl Ester

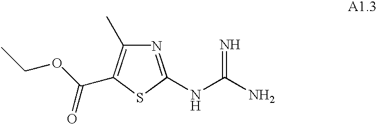

A solution of 2-imino-4-thiobiuret (20.0 g, 0.17 mol), 2-chloroacetoacetate ( 28 g, 0.17 mol) in ethanol (500 mL) was heated to 100° C. for 4 hours. The reaction mixture was concentrated to half volume and poured into 1 liter of 1N NaOH. The white solid which precipitated out was collected by filtration and dried under vacuum to yield A1.3 (30.5 g, 79%). ¹H—NMR (DMSO-d₆) δ:4.22 (2H, q, J=7 Hz), 2.50 (3H, merge with DMSO), 1.26 (3H, t, J=7 Hz). HPLC: 97.7%, ret. time=1.619 min., LC/MS (M+H)⁺=229.

A1.4: 2-(4-Methyl-5-ethoxycarbonylthiazol-2-ylamino)-5,6,7,8-tetrahydro-6-methyl pyrido[4,3-d]pyrimidin-4-ol

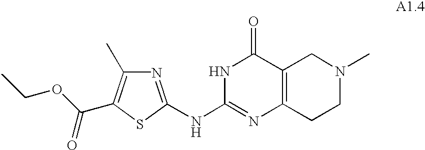

A solution of A1.2 (125 mg, 0.731 mmol), A1.3 (167 mg, 0.731 mmol) and sodium ethoxide(21% in ethanol, 0.989 ml, 2.65 mmol)in DMA was heated to 100° C. for 1 hr and then it was cooled down to RT. The reaction mixture was diluted with 2 mL of water, and neutralized with 1 N HCl. The solid was collected by filtration and dried to yield A1.4 (150 mg, 59%).

A1.5: 2-(4-Methyl-5-ethoxycarbonylthiazol-2-ylamino), 4-chloro-5,6,7,8-tetrahydro-6-methyl-pyrido[4,3-d]pyrimidine

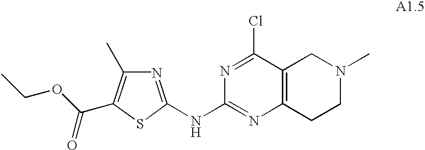

A solution of A1.4 (150 mg, 0.429 mmol) in POCl₃ (1 ml) was heated to 100° C. for 2 hours and then it was cooled down to RT which was poured into 10 ml of ice-water. It was neutralized with NaOH to pH about 9. The solid was collected with filtration and then it was added to 10 ml of methanol and stirred about 10 minutes. The solid was filtered off. The mother solution was concentrated to yield the desired product A1.5 (70 mg, 44.3%). LC/MS (M+H)⁺=368.

A1.6: ′2-[[4-[[[4-(Methylsulfonyl)phenyl]methyl]amino]-5,6,7,8-tetrahydro-6-methylpido[4,3-d]pyrimidin-2-yl]amino]-4-methyl-5-thiazolecarboxylic Acid Ethyl Ester A solution of A1.5 (70 mg, 0.19 mmol) and 4-methylsulfonylbenzylamine hydrochloric salt (66 mg, 0.285 mmol), diisopropylethylamine (111 mg, 0.855 mmol) in N-methyl-2-pyrrolidine (2 mL) was heated to 120 to 130° C. for two hours. The reaction mixture was concentrated to yield a crude product which was purified with prep. HPLC (reverse phase) to yield A1(38 mg, 32% ). ¹H-NMR (CD₃OD) δ:7.78 (2H, d, J=8 Hz), 7.52 (22H, d, J=8 Hz), 4.92 (2H, s), 4.17 (2H, q, JJ=7 Hz), 4.03 (2H, m ), 3.45 (211H, m), 2.93-2.98(81,8m), 2.40 (3H, s), 1.18 (3H, t, J=7 Hz). HPLC: 98%, ret. time=1.58 min., LC/MS (M+H)⁺=517.

### Example A2-A23

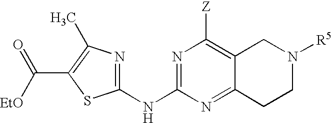

Examples A2 was prepared in a similar manner to that used for Example A1. Example A3 and A4 were prepared in a similar manner to example A1 except intermediate A1.2 was replaced with commercially available methyl 1-benzyl-4-oxo-3-piperdine carboxylate hydrochloride and methyl 4-oxo-3-piperidine carboxylate hydrochloride, and reacted with the appropriate amine corresponding the R¹ group. The R² group was installed after removal of the benzyl group in a manner analogous to that described in the synthesis of example C4, followed by reaction with appropriate reagents.

TABLE A1

| | | | | HPLC | |
|---|---|---|---|---|---|
| | | | | Retention | MS |
| Ex. | Z | R⁵ | Name | (min) | Reported |
| A2 | 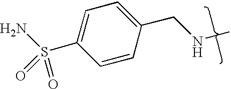 | —Me | 2-[[4-[[[4- (Aminosulfonyl)phenyl]methyl]amino]-5,6,7,8-tetrahydro- 6-methylpyrido[4,3- d]pyrimidin-2-yl]amino]-4- methyl-5-thiazolecarboxylic acid ethyl ester | 1.467 | 518.12 |
| A3 | 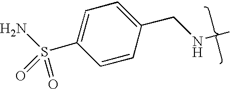 | —Bn | 2-[[4-[[[4- (Aminosulfonyl)phenyl]methyl]amino]-5,6,7,8-tetrahydro- 6-(phenylmethyl)pyrido[4,3- d]pyrimidin-2-yl]amino]-4- methyl-5-thiazolecarboxylic acid ethyl ester | 1.94 | 594.39 |
| A4 | 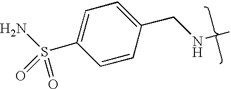 | —H | 2-[[4-[[[4- (Aminosulfonyl)phenyl]methyl]amino]-5,6,7,8- tetrahydropyrido[4,3- d]pyrimidin-2-yl]amino]-4- methyl-5-thiazolecarboxylic acid ethyl ester | 1.46 | 503.59 |
| A5 | 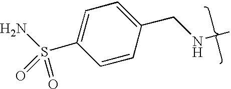 | 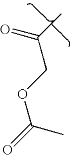 | 2-[[6-[(Acetyloxy)acetyl]-4- [[[4- (aminosulfonyl)phenyl]methyl]amino]-5,6,7,8- tetrahydropyrido[4,3- d]pyrimidin-2-yl]amino]-4- methyl-5-thiazolecarboxylic acid ethyl ester | 2.26 | 604.15 |
| A6 | 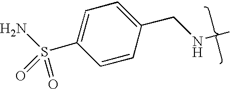 | 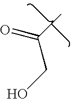 | 2-[[4-[[[4- (Aminosulfonyl)phenyl]methyl]amino]-5,6,7,8-tetrahydro- 6-(hydroxyacetyl)pyrido[4,3- d]pyrimidin-2-yl]amino]-4- methyl-5-thiazolecarboxylic acid ethyl ester | 2.09 | 562.37 |
| A7 | 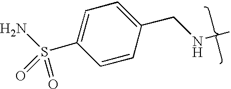 | 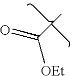 | 2-[[4-[[[4- (Aminosulfonyl)phenyl]methyl]amino]-5,6,7,8-tetrahydro- 6- (ethoxycarbonyl)pyrido[4,3- d]pyrimidin-2-yl]amino]-4- methyl-5-thiazolecarboxylic acid ethyl ester | 1.45 | 576.48 |
| A8 | 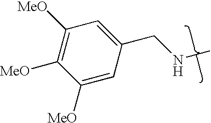 | 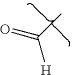 | 2-[[4-[[[3,4,5- Trimethoxyphenyl]methyl]amino]-5,6,7,8-tetrahydro-6- (formyl)pyrido[4,3- d]pyrimidin-2-yl]amino]-4- methyl-5-thiazolecarboxylic acid ethyl ester | 1.48 | 543.47 |
| A9 | 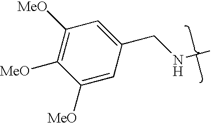 | 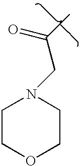 | 2-[[4-[[[3,4,5- Trimethoxyphenyl]methyl]amino]-5,6,7,8-tetrahydro-6- (morpholin-4- ylmethylcarbonyl)pyrido[4,3- d]pyrimidin-2-yl]amino]-4- methyl-5-thiazolecarboxylic acid ethyl ester | 1.36 | 642.48 |
| A10 | 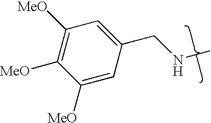 | 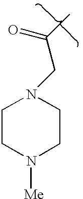 | 2-[[4-[[[3,4,5- Trimethoxyphenyl]methyl]amino]-5,6,7,8-tetrahydro-6-(4- methylpiperazin-1- ylmethylcarbonyl)pyrido[4,3- d]pyrimidin-2-yl]amino]-4- methyl-5-thiazolecarboxylic acid ethyl ester | 1.38 | 655.48 |
| A11 | 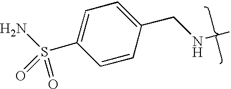 | 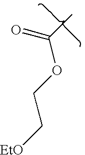 | 2-[[4-[[[4- (Aminosulfonyl)phenyl]methyl]amino]-5,6,7,8-tetrahydro- 6-((2-ethoxy) ethoxycarbonyl)pyrido[4,3- d]pyrimidin-2-yl]amino]-4- methyl-5-thiazolecarboxylic acid ethyl ester | 1.47 | 620.41 |
| A12 | 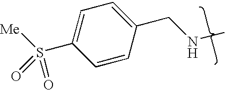 | 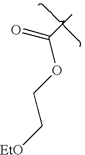 | 2-[[4-[[[4- (Methylsulfonyl)phenyl]methyl]amino]-5,6,7,8-tetrahydro- 6-((2-ethoxy) ethoxycarbonyl)pyrido[4,3- d]pyrimidin-2-yl]amino]-4- methyl-5-thiazolecarboxylic acid ethyl ester | 1.49 | 619.42 |
| A13 | 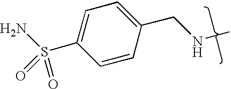 | —CN | 2-[[4-[[[4- (Aminosulfonyl)phenyl]methyl]amino]-5,6,7,8-tetrahydro- 6-(cyano)pyridol[4,3- d]pyrimidin-2-yl]amino]-4- methyl-5-thiazolecarboxylic acid ethyl ester | 1.27 | 529.44 |
| A14 | 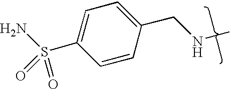 | 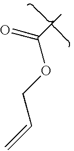 | 2-[[4-[[[4- (Aminosulfonyl)phenyl]methyl]amino]-5,6,7,8-tetrahydro- 6- (allyloxycarbonyl)pyrido[4,3- d]pyrimidin-2-yl]amino]-4- methyl-5-thiazolecarboxylic acid ethyl ester | 1.50 | 588.43 |
| A15 | 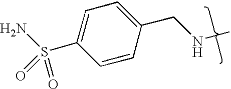 | 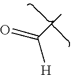 | 2-[[4-[[[4- (Aminosulfonyl)phenyl]methyl]amino]-5,6,7,8-tetrahydro- 6- (allyloxycarbonyl)pyrido[4,3- d]pyrimidin-2-yl]amino]-4- methyl-5-thiazolecarboxylic acid ethyl ester | 1.23 | 532.42 |
| A16 | 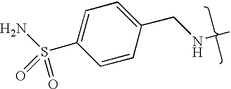 | 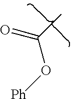 | 2-[[4-[[[4- (Aminosulfonyl)phenyl]methyl]amino]-5,6,7,8-tetrahydro- 6- (phenyloxycarbonyl)pyrido[4, 3-d]pyrimidin-2-yl]aminol-4- methyl-5-thiazolecarboxylic acid ethyl ester | 1.56 | 624.42 |
| A17 | 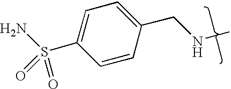 | 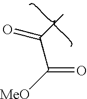 | 2-[[4-[[[4- (Aminosulfonyl)phenyl]methyl]amino]-5,6,7,8-tetrahydro- 6- (methoxycarbonylcarbonyl) pyrido[4,3-d]pyrimidin-2- yl]amino]-4-methyl-5- thiazolecarboxylic acid ethyl ester | 1.34 | 590.44 |
| A18 | 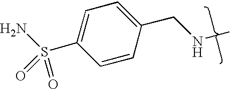 | 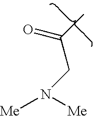 | 2-[[4-[[[4- (Aminosulfonyl)phenyl]methyl]amino]-5,6,7,8-tetrahydro- 6- (dimethylaminomethylcarbonyl) pyrido[4,3-d]pyrimidin-2- yl]amino]-4-methyl-5- thiazolecarboxylic acid ethyl ester | 1.12 | 589.48 |
| A19 | 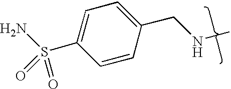 | 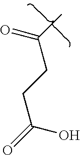 | 2-[[4-[[[4- (Aminosulfonyl)phenyl]methyl]amino]-5,6,7,8-tetrahydro- 6- (carboxyethylcarbonyl)pyrido [4,3-d]pyrimidin-2- yl]amino]-4-methyl-5- thiazolecarboxylic acid ethyl ester | 1.27 | 604.43 |
| A20 | 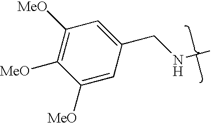 | 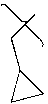 | 2-[[4-[[[3,4,5- Trimethoxyphenyl]methyl]amino]-5,6,7,8-tetrahydro-6- (cyclopropylmethyl)pyrido[4, 3-d]pyrimidin-2-yl]amino]-4- methyl-5-thiazolecarboxylic acid ethyl ester | 1.63 | 569.57 |
| A21 | 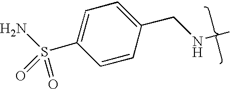 | 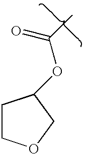 | 2-[[4-[[[4- (Aminosulfonyl)phenyl]methyl]amino]-5,6,7,8-tetrahydro- 6-((3- (tetrahydrofuranyl)oxycarbonyl) pyrido[4,3-d]pyrimidin-2- yl]amino]-4-methyl-5- thiazolecarboxylic acid ethyl ester | 1.38 | 618.46 |
| A22 | 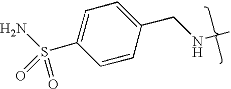 | 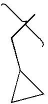 | 2-[[4-[[[4- (Aminosulfonyl)phenyl]methyl]amino]-5,6,7,8-tetrahydro- 6- (cyclopropylmethyl)pyrido[4, 3-d]pyrimidin-2-yl]amino]-4- methyl-5-thiazolecarboxylic acid ethyl ester | 1.10 | 558.49 |
| A23 | 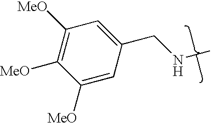 | 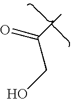 | 2-[[4-[[[3,4,5- Trimethoxyphenyl]methyl]amino]-5,6,7,8-tetrahydro-6- (hydroxyacetyl)pyrido[4,3 - d]pyrimidin-2-yl]amino]-4- methyl-5-thiazolecarboxylic acid ethyl ester | 1.44 | 573.51 |
| A24 | 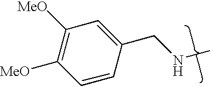 | 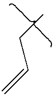 | 2-[[4-[[[3,4,- Dimethoxyphenyl]methyl]amino]-5,6,7,8-tetrahydro-6-(2- propenyl)pyrido[4,3- d]pyrimidin-2-yl]amino]-4- methyl-5-thiazolecarboxylic acid ethyl ester | 1.16^{a} | 525.31 |
| A25 | 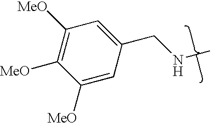 | 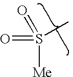 | 2-[[4-[[[3,4,5- Trimethoxyphenyl]methyl]amino]-5,6,7,8-tetrahydro-6- (methylsulfonyl)pyrido[4,3- d]pyrimidin-2-yl]amino]-4- methyl-5-thiazolecarboxylic acid ethyl ester | 1.26^{a} | 593.21 |

^{a}HPLC conditions used to determine retention times; 2 min gradient 0-100% B in A(A; 0.1% TFA in 90/10 water/methanol; B; 0.1% TFA in 10/90 water/methanol) using a Phenomenex S5 ® colunm at 254 nm.

Example A26-A28

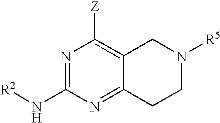

The compounds in Table A2 were prepared using the appropriate guanidine corresponding to A1.3. A26 and A27 were elaborated as described for A3. A28 was elaborated as described for the synthesis of example C4.1 with the exception that benzylchloroformate was replaced with ethylchoroformate.

TABLE A2

| | | | | | HPLC | |
|---|---|---|---|---|---|---|
| | | | | | Retention | MS |
| Ex. | Z | R⁵ | R² | Name | (min) | Reported |
| A26 | 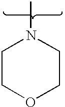 | —Bn | 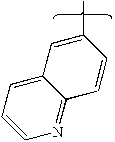 | 6-[[4-morpholinyl-5,6,7,8- tetrahydro-6- (phenylmethyl)pyrido[4,3- d]pyrimidin-2-yl]amino]- quinoline | 0.77 | 454.35 |
| A27 | 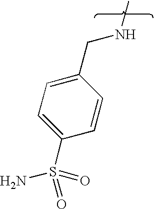 | —Bn | 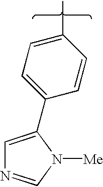 | 1-[[4-[[[4- (Aminosulfonyl)phenyl]methyl]amino]-5,6,7,8-tetrahydro- 6-(phenylmethyl)pyrido[4,3- d]pyrinidin-2-yl]amino]-4- [(1-methyl)imidazol- 5yl]benzene | 0.82 | 581.52 |
| A28 | 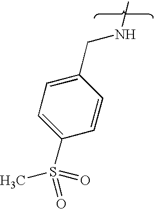 | 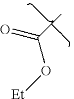 | 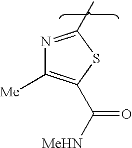 | 2-[[4-[[[4- (Methylsulfonyl)phenyl]methyl]amino]-5,6,7,8-tetrahydro- 6- (ethoxycarbonyl)pyrido[4,3- d]pyrimidin-2-yl]amino]-4- methyl-5-thiazolecarboxylic acid methyl amide | 1.17 | 560.46 |

HPLC conditions used to determine retention times; 2 min gradient 0-100% B in A(A; 0.1% TFA in 90/10 water/methanol; B; 0.1% TFA in 10/90 water/methanol) using a Phenomenex S5 ® column at 254 nm.

Example B1

### 2-[[4-[[[4-(Aminosulfony)phenyl]methyl]amino]-6,7,8,9-tetrahydro-5H-pyrimido[4,5-d]azepin-2-yl]amino]-4-methyl-5-thiazolecarboxylic acid ethyl ester

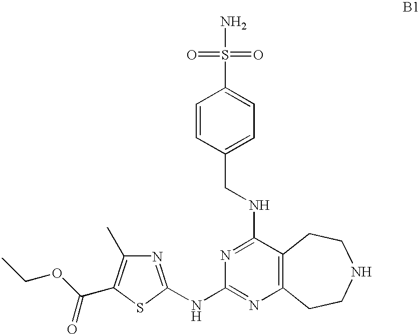

B1.1: Hexahydro-5-oxo-1H-Azepine-1,4-dicarboxylic acid 4-tertbutyl 1-methyl ester

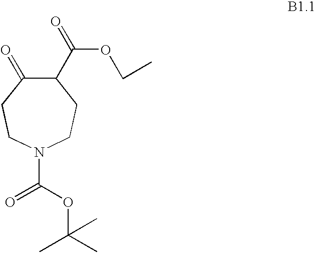

A solution of commercially available N-tertbutoxycarbonyl-4-piperidone (500 mg, 2.46 mmol) in 2 mL of ethyl ether (2 mL) was simultaneously added boron trifluoride etherate (349 mg, 2.46 mmol) and ethyl diazoacetate dropwise (371 mg, 3.25 mmol) at −25° C. to −30° C. The reaction mixture was maintianed at −25° C. to −30° C. for one hour and then it was warmed to RT. The reaction mixture was diluted with ethyl ether (30 ml) and was washed with saturated Na₂CO₃ solution (20 mL) and the organic layer dried over sodium sulfate. Filtration and concentration to yield a crude product which was purified on silica gel column with dichloromethane/methanol (50/1 to 20/1) to yield B1.1 (662 mg, 94.4%). HPLC: 91%.

B1.2: 2-(4-Methyl-5-ethoxycarbonylthiazol-2-ylamino)-5,6,8,9-tetrahydro-7-tertbutyloxycarbonylpprido[4,5-d]az(pin-4-ol

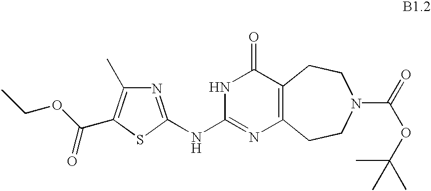

A solution of A1.3 (110 mg, 0.485 mmol) and sodium ethoxide (21% in ethanol, 0.656 ml, 1.76 mmol) in ethanol (2 ml) was heated to 100° C. for half an hour and then it was cooled down to RT which was added B1.1 (138 mg, 0.485 mmol). The reaction mixture was heated to 100° C. for 2 days. It was concentrated to yield a crude product which was diluted with 2 mL of water and neutralized with 1 N HCl. The solid was collected by filtrationand stirred with anhydrous methanol for 10 minutes. The resulting solid was collected by filtration to yield B1.2 (77 mg, 35%). LC/MS (M+H)⁺=450.

B1.3: 4-Chloro-2-(4-methyl-5-ethoxycarbonylthiazol-2-ylamino)-5,6,8,9-tetrahydro-7H— pyrido[4,5-d]azepine

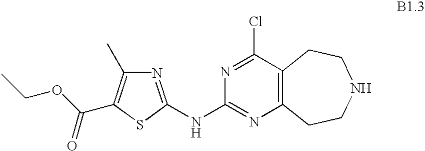

A solution of B 1.2 (77 mg, 0.172 mmol) in POCl₃ (0.5 ml) was heated to 100° C. for 16 hours and then it was cooled down to RT which was poured into 5 ml of ice-water. It was neutralized with NaOH to pH about 9. The solid was collected by filtration and then it was added to 3 mL of methanol and stirred about 20 minutes. The solid was collected to yield B1.3 (67 mg). LC/MS (M+H)⁺=368.

B1.4: 2-[[4-[[[4-(Aminosulfonyl)phenyl]methyl]amino]-6,7,8,9-tetrahydro-5H-pyrimido[4,5-d]azepin-2-yl]amino]-4-methyl-5-thiazolecarboxylic Acid Ethyl Ester

A solution of B1.3 (20 mg, 0.0544 mmol) and p-aminomethylbenzenesulfonamide hydrochloric salt (24 mg, 0.109 mmol), diisopropylethylamine (57 uL, 0.326 mmol) in N-methyl-2-pyrrolidine (0.5 ml) was heated to 120 to 130° C. for an hour. The reaction mixture was concentrated to yield a crude product which was purified with prep. HPLC ( reverse phase) to yield B1 (2.5 mg, 9% ). ¹H—NMR (CD₃OD) δ:7.86 (2H, d, J=8 Hz ), 7.56 (2H, d, J=8 Hz), 5.09 (2H, s), 4.31 (2H, q, JJ=7 Hz), 3.44-3.45 (4H, m), 3.20-3.26 (4H, m), 3.08-3.14 (4H, m), 2.54 (3H, s), 1.32 (3H, t, J=7 Hz). HPLC: 98%, ret. time=1.593 min., LC/MS (M+H)⁺=518.

### Example B2-B4

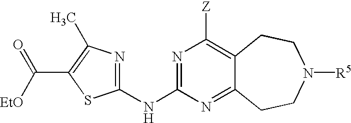

Examples B2 to B4 were prepared in a similar manner to that used for Example B1, with the exception that the 7-amine position was reacted with an appropriate acid chloride.

TABLE B

| | | | | HPLC | |
|---|---|---|---|---|---|
| | | | | Retention | MS |
| Ex. | Z | R⁵ | Name | (min) | Reported |
| B2 | 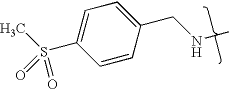 | 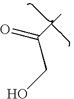 | 4-Methyl-2-[[6,7,8,9- tetrahydro-7-(hydroxyacetyl)- 4-[[[4- (methylsulfonyl)phenyl]methyl]amino]-5H-pyrimido[4,5- d]azepin-2-yl]amino]-5- thiazolecarboxylic acid ethyl ester | 2.19 | 575.13 |
| B3 | 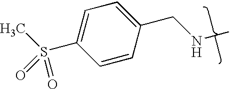 | 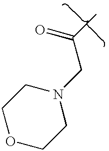 | 4-Methyl-2-[[6,7,8,9- tetrahydro-4-[[[4- (methylsulfonyl)phenyl]methyl]amino]-7-(4- morpholinylacetyl)-5H- pyrimido[4,5-d]azepin-2- yl]amino]-5- thiazolecarboxylic acid ethyl ester | 1.77 | 644.16 |
| B4 | 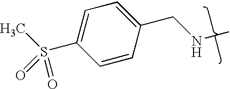 | 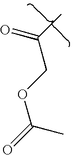 | 2-[[7-[(Acetyloxy)acetyl]- 6,7,8,9-tetrahydro-4-[[[4- (methylsulfonyl)phenyl]methyl]amino]-5H-pyrimido[4,5- d]azepin-2-yl]amino]-4- methyl-5-thiazolecarboxylic acid ethyl ester | 2.30 | 617.15 |

### Example C1

### 4-Methyl-2-[[5,6,7,8-tetrahydro-7-(phenylmethyl)-4-(1-piperazinyl)pyrido[3,4-d]pyrimidin-2-yl]amino]-5-thiazolecarboxylic Acid Ethyl Ester

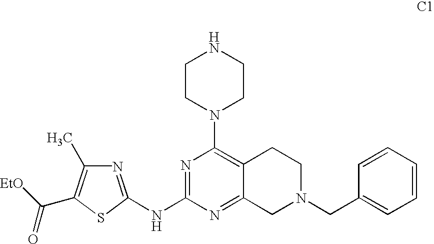

C1.1: 2-(4-Methyl-5-ethoxycarbonylthiazol-2-ylamino)-5,6,7,8-tetrahydro-7-(phenylmethyl)pyrido[3,4-d]pyrimidin-4-ol

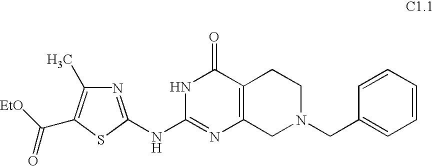

A solution of ethyl 1-benzyl-3-oxo-piperidinecarboxylate.HCl (2.90 g, 9.74 mmol), A1.3 (2.0 g, 8.8 mmol) and sodium ethoxide (21% in ethanol, 13.1 ml, 35.2 mmol) in ethanol (40 ml) was heated to 100° C. for 2 hrs and then it was cooled down to RT which was concentrated to yield a crude product. It was added 100 ml of water which was neutralized with 1 N HCl until PH about 7. The solid was collected by filtration and dried under vacuum to yield C1.1 (3.14 g, 84%). LC/MS (M+H)⁺=426.48.

C1.2: 2-(4-Methyl-5-ethoxycarbonylthiazol-2-ylamino), 4-chloro-5,6,7,8-tetrahydro-7-(phenylmethyl)pyrido[3,4-d]pyrimidine

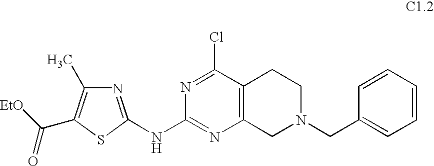

A solution of C1.1 (3.14 g, 7.38 mmol) in POCl₃ (25 ml) was heated to 100° C. for 1 hour and then it was cooled down to RT which was poured into 100 ml of ice-water. The reaction mixture was neutralized with 1 N sodium hydroxide to about pH 9. The solid was filtered and dried under vacuum to yield C1.2 (2.80 g, 86% ). LC/MS (M+H)⁺=444.08.

C1.3: 4-Methyl-2-[[5,6,7,8-tetrahydro-7-(phenylmethyl)-4-(4-tertbutloxycarbonyl-1-piperazinyl)pyrido[3,4-d]pyrimidin-2-yl]amino]-5-thiazolecarboxylic Acid Ethyl Ester

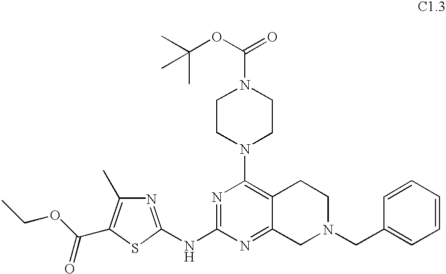

A solution of C1.2 (100 mg, 0.225 mmol) and 1-tertbutyloxycarbonylpiperazine (45 mg, 0.236 mmol), diisopropylethylamine (0.137 ml, 0.785 mmol) in N-methyl-2-pyrrolidine (1 mL) was heated to 120 to 130° C. for one hour. The reaction mixture was concentrated under reduced pressure to yield a crude product which was added 2 mL of methanol and stirred for 10 minutes during which time a solid precipitated. The solid was collected by filtration and dried under vacuum to yield C1.3 (66 mg, 49% ). ¹H—NMR (DMSO) δ: 7.26-7.39 (5H, m ), 4.21 (2H, q, J=7 Hz), 3.67 (2H, s ), 2.57-3.60 (14H, m ), 2.50 (3H, merge with DMSO), 1.42 (9H, s ), 1.29 (3H, t, J=7 Hz). HPLC: 90%, ret. time=3.24 min., LC/MS (M+H)⁺=594.20.

C1.4: 4-Methyl-2-[[5,6,7,8-tetrahydro-7-(phenylmethyl)-4-(1-piperazinyl)pyrido[3,4-d]pyrimidin-2-yl]amino]-5-thiazolecarboxylic Acid Ethyl Ester

To a solution of C1.3 (40 mg, 0.0674 mmol) in dichloromethane (0.5 mL) was added trifluoroacetic acid (0.5 mL) which was stirred at RT for half an hour. The reaction mixture was concentrated to yield a crude product which was added 5 mL of ethyl ether. The solid was collected and dried under vacuum to yield C1 (46.6 mg, 99%). ¹H—NMR (CD₃OD) δ: 7.26-7.44 (5H, m ), 4.16-4.28 (44H, m), 3.67 (2H, s), 2.00-4.00 (17H, m ), 1.24 (3H, t, J=7 Hz). HPLC: 96%, ret. time=1.92 min., LC/MS (M+H)+=494.15.

### Example C₂-C₃

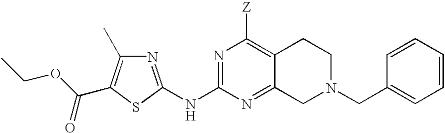

Examples C2 to C3 were prepared in a similar manner to that used for Example C1 using appropriate reagents.

TABLE C1

| | | | HPLC | |
|---|---|---|---|---|
| | | | Retention^{a} | MS |
| Ex. | Z | Name | (min) | Reported |
| C2 | 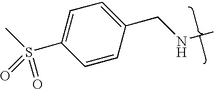 | 4-Methyl-2-[[5,6,7,8-tetrahydro-7- (phenylmethyl)-4-[4- (methylsulfonyl)phenyl]methyl]amino]pyrido[3,4-d]pyrimidin-2-yl]amino]- 5-thiazolecarboxylic acid ethyl ester | 2.32 | 593.10 |
| C3 | 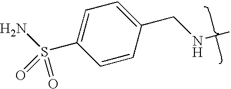 | 4-Methyl-2-[[5,6,7,8-tetrahydro-7- (phenylmethyl)-4-[4- (aminosulfonyl)phenyl]methyl]amino]pyrido[3,4-d]pyrimidin-2-yl]amino]- 5-thiazolecarboxylic acid ethyl ester | 2.24 | 594.30 |

^{a}HPLC conditions used to determine retention times; 4 min gradient 0-100% B in A(A; 0.1% TEA in 90/10 water/methanol; B; 0.1% TFA in 10/90 water/methanol) using a YMC turbopack colunm at 254 nm.

### Example C4

4-Methyl-2-[[5,6,7,8-tetrahydro-7-((3,4-(dimethoxy)phenyl)methyl)-4-(1-piperazinyl)pyrido[3,4-d]pyrimidin-2-yl]amino]-5-thiazolecarboxylic Acid Ethyl Ester

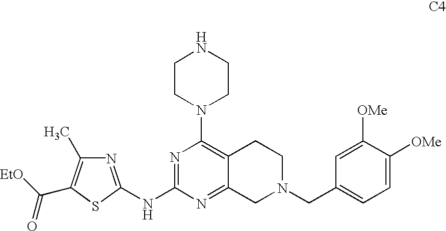

C4.1: 4-Methyl-2-[[5,6,7,8-tetrahydro-7-benzyloxycarbonyl-4-(4-tertbutyloxycarbony-1-piperazinyl)pyrido[3,4-d]pyrimidin-2-yl]amino]-5-thiazolecarboxylic Acid Ethyl Ester

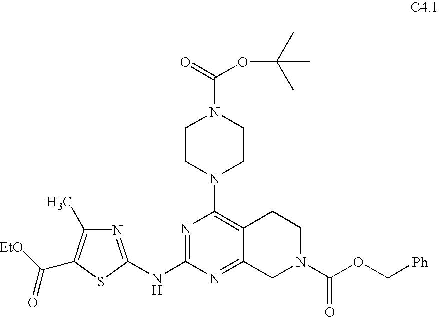

C1.3 (250 mg, 0.42 mmol) was dissolved in dichloroethane, and benzyl chloroformate (200 mg, 1.1 mmol) was added and the reaction mixture refluxed overnight. The reaction mixture was concentrated and purified by silica gel column chromatography to yield C4.1 (220 mg, 82%). (M+H)⁺=638.51.

C4.2: 4-Methyl-2-[[5,6,7,8-tetrahydro-4-(4-tertbutyloxycarbonyl-1-piperazinyl)pyrido[3,4-d]pyrimidin-2-yl]amino]-5-thiazolecarboxylic Acid Ethyl Ester

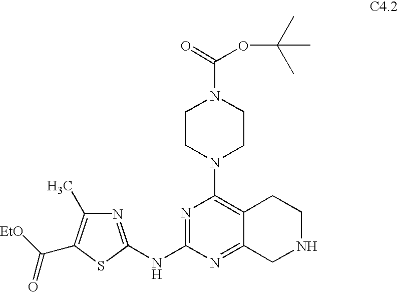

C4.1 (210 mg, 0.33 mmol) was dissolved in 10 mL of acetic acid and 220 mg of 10% palladium on carbon was cautiously added under an inert atmosphere. The reaction mixture was hydrogenated overnight (18 h) at 50 psi using a Parr apparatus. The reaction mixture was filtered, concentrated and purified by prep HPCL to yield C4.2 (114 mg, 70%) as an oil. ¹H—NMR (CD₃OD) δ: 4.15-4.25 (44H, m), 3.50-3.65 (8H, m), 3.50 ( 2H, m), 2.92 (2H,), 2.48 (3H, s), 1.40 (9H, s), 1.27 (3H, t, J=7 Hz). (M+H)⁺=504.18.

C4.3: 4-Methyl-2-[[5,6,7,8-tetrahydro-7-((3,4-(dimethoxy)phenyl)methyl)-4-(4-tertbutyloxycarbonyl-1-piperazinyl)pyrido[3,4-d]pyrimidin-2-yl]amino]-5-thiazolecarboxylic Acid Ethyl Ester

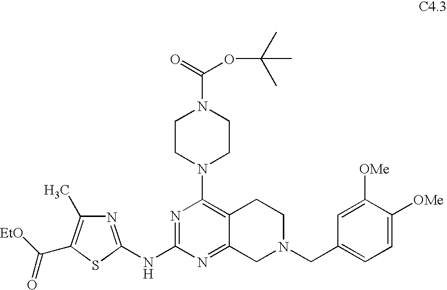

3,4-Dimethoxybenzaldehyde (7 mg, 0.040 mmol), C4.2 (20 mg, 0.040 mmol), and triacetoxyborohydride (17 mg, 0.077 mmol) were suspended in dichloroethane, and stirred at room temperature overnight. The reaction mixture was concentrated, and purified by preparatory HPLC to yield C4.3 (22 mg, 85%). ¹H—NMR (CDCl₃) δ: 6.60-6.80 (3H, m), 4.05-4.25 (6H, m ), 3.71 (3H, s), 3.72 (3H, s) 3.50-3.65 (8H, m ), 2.55-3.45 (4H, m), 2.50 (3H, s), 1.30 (9H, s), 1.22 (3H, t, J=7 Hz). (M+H)⁺=654.25.

C4.4: 4-Methyl-2-[[5,6,7,8-tetrahydro-7-((3,4-(dimethoxy)phenyl)methyl)-4-(1-piperazinyl)pyrido[3,4-d]pyrimidin-2-yl]amino]-5-thiazolecarboxylic Acid Ethyl Ester

C4.3 (14 mg, 0.021 mmol) was dissolved in 0.5 mL of trifluoroacetic acid and stirred at room temperature for 0.5 h. The solvent was evaporated to provided C4.4 (12 mg, 100%). ¹H—NMR (CD₃OD) δ: 6.85-7.05 (33H, m ), 4.33 (2H, s), 4.05-4.20 (44H, m ), 3.75 (3H, s), 3.73 (3H, s) 3.65 (4H, m), 3.16 (2H, merge with CD₃OD)), 2.85 (2H, m), 2.42 (3H, s), 1.22 (3H, t, J=7 Hz). (M+H)⁺=554.49.

### Example C₅-C₂₄

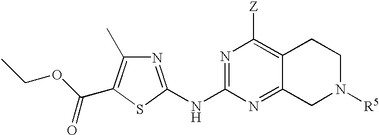

Examples C5 to C24 were prepared in a similar manner to that used for Example C4 using appropriate reagents.

TABLE C1

| | | | | HPLC | |
|---|---|---|---|---|---|
| | | | | Retention | MS |
| Ex. | R⁵ | Z | Name | (min) | Reported |
| C5 | 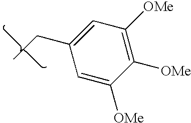 | 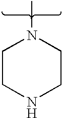 | 4-Methyl-2-[[5,6,7,8- tetrahydro-7-((3,4,5- (trimethoxy)phenyl)methyl)- 4-(1-piperazinyl)pyrido[3,4- d]pyrimidin-2-yl]amino]-5- thiazolecarboxylic acid ethyl ester | 1.94 | 584.18 |
| C6 | 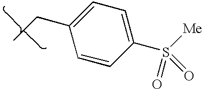 | 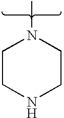 | 4-Methyl-2-[[5,6,7,8- tetrahydro-7-((4- (methylsulfonyl)phenyl)methyl)- -4-(1- piperazinyl)pyrido[3,4- d]pyrimidin-2-yl]amino]-5- thiazolecarboxylic acid ethyl ester | 1.69 | 572.16 |
| C7 | 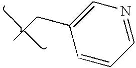 | 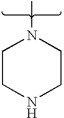 | 4-Methyl-2-[[5,6,7,8- tetrahydro-7-((3- pyridyl)methyl)-4-(1- piperazinyl)pyrido[3,4- d]pyrimidin-2-yl]amino]-5- thiazolecarboxylic acid ethyl ester | 1.31 | 495.13 |
| C8 | 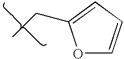 | 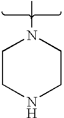 | 4-Methyl-2-[[5,6,7,8- tetrahydro-7-((2- furanyl)methyl)-4-(1- piperazinyl)pyrido[3,4- d]pyrimidin-2-yl]amino]-5- thiazolecarboxylic acid ethyl ester | 1.75 | 484.14 |
| C9 | 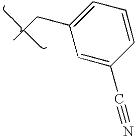 | 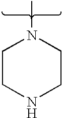 | 4-Methyl-2-[[5,6,7,8- tetrahydro-7-((3- (cyano)phenyl)methyl)-4-(1- piperazinyl)pyrido[3,4- d]pyrimidin-2-yl]amino]-5- thiazolecarboxylic acid ethyl ester | 1.87 | 519.16 |
| C10 | 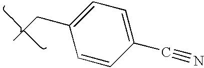 | 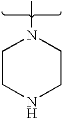 | 4-Methyl-2-[[5,6,7,8- tetrahydro-7-((4- (cyano)phenyl)methyl)-4-(1- piperazinyl)pyrido[3,4- d]pyrimidin-2-yl]amino]-5- thiazolecarboxylic acid ethyl ester | 1.91 | 519.35 |
| C11 | 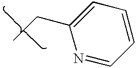 | 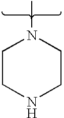 | 4-Methyl-2-[[5,6,7,8- tetrahydro-7-((2- pyridyl)methyl)-4-(1- piperazinyl)pyrido[3,4- d]pyrimidin-2-yl]amino]-5- thiazolecarboxylic acid ethyl ester | 1.49 | 495.19 |
| C12 | 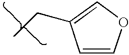 | 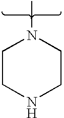 | 4-Methyl-2-[[5,6,7,8- tetrahydro-7-((3- furanyl)methyl)-4-(1- piperazinyl)pyrido[3,4- d]pyrimidin-2-yl]amino]-5- thiazolecarboxylic acid ethyl ester | 1.67 | 484.21 |
| C13 | 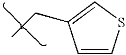 | 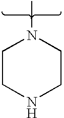 | 4-Methyl-2-[[5,6,7,8- tetrahydro-7-((3- thenyl)methyl)-4-(1- piperazinyl)pyrido[3,4- d]pyrimidin-2-yl]amino]-5- thiazolecarboxylic acid ethyl ester | 1.84 | 500.17 |
| C14 | 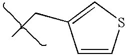 | 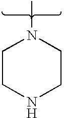 | 4-Methyl-2-[[5,6,7,8- tetrahydro-7-((4- pyridyl)methyl)-4-(1- piperazinyl)pyrido[3,4- d]pyrimidin-2-yl]amino]-5- thiazolecarboxylic acid ethyl ester | 1.34 | 495.19 |
| C15 | 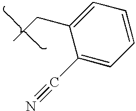 | 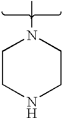 | 4-Methyl-2-[[5,6,7,8- tetrahydro-7-((2- (cyano)phenyl)methyl)-4-(1- piperazinyl)pyrido[3,4- d]pyrimidin-2-yl]amino]-5- thiazolecarboxylic acid ethyl ester | 2.13 | 519.23 |
| C16 | 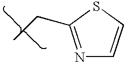 | 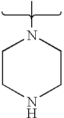 | 4-Methyl-2-[[5,6,7,8- tetrahydro-7-((2- thiazolyl)methyl)-4-(1- piperazinyl)pyrido[3,4- d]pyrimidin-2-yl]amino]-5- thiazolecarboxylic acid ethyl ester | 1.84 | 501.49 |
| C17 | 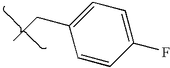 | 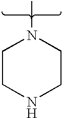 | 4-Methyl-2-[[5,6,7,8- tetrahydro-7-((4- fluorophenyl)methyl)-4-(1- piperazinyl)pyrido[3,4- d]pyrimidin-2-yl]amino]-5- thiazolecarboxylic acid ethyl ester | 1.98 | 512.33 |
| C18 | 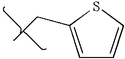 | 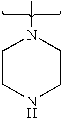 | 4-Methyl-2-[[5,6,7,8- tetrahydro-7-((2- thenyl)methyl)-4-(1- piperazinyl)pyrido[3,4- d]pyrimidin-2-yl]amino]-5- thiazolecarboxylic acid ethyl ester | 1.88 | 500.31 |
| C19 | 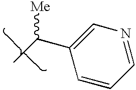 | 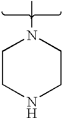 | 4-Methyl-2-[[5,6,7,8- tetrahydro-7-(1-(3- pyridyl)ethyl)-4-(1- piperazinyl)pyrido[3,4- d]pyrimidin-2-yl]amino]-5- thiazolecarboxylic acid ethyl ester | 1.41 | 509.48 |
| C20 | 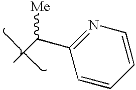 | 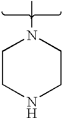 | 4-Methyl-2-[[5,6,7,8- tetrahydro-7-(1-(2- pyridyl)ethyl)-4-(1- piperazinyl)pyrido[3,4- d]pyrimidin-2-yl]amino]-5- thiazolecarboxylic acid ethyl ester | 1.64 | 509.18 |
| C21 | 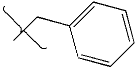 | 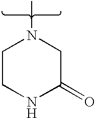 | 4-Methyl-2-[[5,6,7,8- tetrahydro-7-(phenyl)methyl)- 4-(3-oxo-1- piperazinyl)pyrido[3,4- d]pyrimidin-2-yl]amino]-5- thiazolecarboxylic acid ethyl ester | 2.28 | 508.35 |
| C22 | 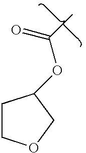 | 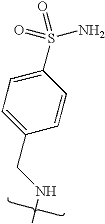 | 4-Methyl-2-[[5,6,7,8- tetrahydro-7- ((tetrahydrofuran-3- yl)oxycarbonyl)-4-[4- (aminosulfonyl)phenyl]methyl]amino]pyrido[3,4- d]pyrimidin-2-yl]amino]-5- thiazolecarboxylic acid ethyl ester | 1.40 | 618.47 |
| C23 | 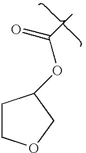 | —OBu | 4-Methyl-2-[[5,6,7,8- tetrahydro-7- ((tetrahydrofuran-3- yl)oxycarbonyl)-4- (butyloxy)pyrido[3,4- d]pyrimidin-2-yl]amino]-5- thiazolecarboxylic acid ethyl ester | 2.02 | 506.51 |
| C24 | 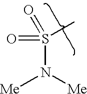 | 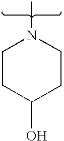 | 4-Methyl-2-[[5,6,7,8- tetrahydro-7- (dimethylaminosulfonyl)-4- ((4-hydroxy)-1- piperidinyl)pyrido[3,4- d]pyrimidin-2-yl]amino]-5- thiazolecarboxylic acid ethyl ester | 2.34 | 526.23 |

We claim:

1. A compound of formula I 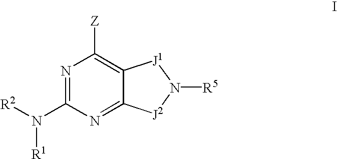
Including enantiomers, diastereomers, and pharmaceutically acceptable salts thereof, wherein
R¹ is hydrogen or alkyl;
R² is
(a) heteroaryl, or heterocyclo, either of which may be optionally substituted with one to three groups T¹, T², T³;
(b) aryl substituted with one to three groups T¹, T², T³ provided that at least one of T¹, T², T³ isother than H; or
(c) aryl fused to a heteroaryl or heterocyclo ring wherein the combined ring system may be optionally substituted with one to three groups T¹, T², T³;
Z is NR³R⁴, NR³SO₂R^{4a}, OR⁴, SR⁴, haloalkyl, or halogen;
R³ and R⁴ are independently H, alkyl, alkenyl, aryl, (aryl)alkyl, heteroaryl, (heteroaryl)alkyl, cycloalkyl, (cycloalkyl)alkyl, heterocylo or (heterocyclo)alkyl any of which may be optionally independently substituted where valance allows with one to three groups T^{1a}, T^{2a} or T^{3a};
or R³ and R⁴ may be taken together with the nitrogen atom to which they are attached to form a heterocyclo or heteroaryl ring optionally independently substituted where valance allows with one to three groups T^{1a}, T^{2a} or T^{3a};
R^{4a} is alkyl, alkenyl, aryl, (aryl)alkyl, heteroaryl, (heteroaryl)alkyl, cycloalkyl, (cycloalkyl)alkyl, heterocylo or (heterocyclo)alkyl any of which may be optionally independently substituted where valance allows with one to three groups T^{1a}, T^{2a} or T^{3a};
R^{3b} and R^{4b} are independently H, alkyl, alkenyl, aryl, (aryl)alkyl, heteroaryl, (heteroaryl)alkyl, cycloalkyl, (cycloalkyl)alkyl, heterocylo or (heterocyclo)alkyl;
R⁵ is
(a) hydrogen, or cyano;
(b) alkyl, alkenyl, alkynyl, cycloalkyl, (cycloalkyl)alkyl, aryl, (aryl)alkyl, heterocyclo, (heterocyclo)alkyl, heteroaryl or (heteroaryl)alkyl, any of which may be optionally independently substituted where valance allows with one to three groups T^{1b}, T^{2b} or T^{3b}; or
(c) —C(O)R⁶, —C(O)OR⁶, —C(O)—C(O)OR , or —SO₂R^{6a};
R⁶ is H, alkyl, alkenyl, —NR^{3b}R^{4b}, heterocylco, (heterocyclo)alkyl, (hydroxy)alkyl, (alkoxy)alkyl, (aryloxy)alkyl, (NR^{3b}R^{4b})alkyl, heteroaryl, aryl or (aryl)alkyl, any of which may be optionally independently substituted where valance allows with one to three groups T^{1b}, T^{2b} or T^{3b};
R^{6a} is alkyl, alkenyl, —NR^{3b}R^{4b}, heterocylco, (heterocyclo)alkyl, (hydroxy)alkyl, (alkoxy)alkyl, (aryloxy)alkyl, (NR^{3b}R^{4b})alkyl, heteroaryl, aryl or (aryl)alkyl, any of which may be optionally independently substituted where valance allows with one to three groups T^{1b}, T^{2b} or T^{3b};
J¹ and J² are independently optionally substituted C₁₋₃ alkylene, provided that J¹ and J² are not both greater than C₂ alkylene; and
T^{1-1b}, T^{2-2b}, and T^{3-3b} are are each independently
(1) hydrogen or T⁶, where T⁶ is
(i) alkyl, (hydroxy)alkyl, (alkoxy)alkyl, alkenyl, alkynyl, cycloalkyl, (cycloalkyl)alkyl, cycloalkenyl, (cycloalkenyl)alkyl, aryl, (aryl)alkyl, heterocyclo, (heterocylco)alkyl, heteroaryl, or (heteroaryl)alkyl;
(ii) a group (i) which is itself substituted by one or more of the same or different groups (i); or
(iii) a group (i) or (ii) which is independently substituted by one or more (preferably 1 to 3) of the following groups (2) to (13) of the definition of T^{1-1b}, T^{2-2b} and T^{3-3b},
(2) —OH or —OT⁶,
(3) —SH or —ST⁶,
(4) —C(O)ₜH, —C(O)ₜT⁶, or —O—C(O)T⁶, where t is 1 or 2,
(5) —SO₃H, —S(O)ₜT⁶, or S(O)ₜN(T⁹)T⁶,
(6) halo,
(7) cyano,
(8) nitro,
(9) —T⁴—NT⁷T⁸,
(10) —T⁴—N(T⁹)—T⁵—NT⁷T⁸,
(11)—T⁴—N(T¹⁰)—T⁵—T⁶,
(12) —T⁴—N(T¹⁰)—T⁵—H,
(13) oxo,
T⁴ and T⁵ are each independently
(1) a single bond,
(2) —T¹¹—S(O)ₜ—T¹²—,
(3) —T¹¹—C(O)—T¹²—,
(4) —T¹¹—C(S)—T¹²—,
(5) —T¹¹—O—T¹²—,
(6) —T¹¹—S—T¹²—,
(7) —T¹¹—O—C(O)—T¹² —,
(8) —T¹¹—C(O)—O—T¹²—,
(9) —T¹¹—C(═NT^{9a})—T¹²—, or
(10) —T¹¹—C(O)—C(O)—T¹²—,
T⁷, T⁸, T⁹, T^{9a} and T¹⁰
(1) are each independently hydrogen or a group provided in the definition of T⁶, or
(2) T⁷ and T⁸ may together be alkylene or alkenylene, completing a 3- to 8-membered saturated or unsaturated ring together with the atoms to which they are attached, which ring is unsubstituted or substituted with one or more groups listed in the description of T^{1-1b}, T^{2-2b} and T^{3-3b}, or
(3) T⁷ or T⁸, together with T⁹, may be alkylene or alkenylene completing a 3- to 8-membered saturated or unsaturated ring together with the nitrogen atoms to which they are attached, which ring is unsubstituted or substituted with one or more groups listed in the description of T^{1-1b}, T^{2-2b} and T^{3-3b}, or
(4) T⁷ and T⁸ or T⁹ and T¹⁰ together with the nitrogen atom to which they are attached may combine to form a group —N═CT¹³T¹⁴ where T¹³ and T¹⁴ are each independently H or a group provided in the definition of T⁶; and
T¹¹ and T¹² are each independently
(1) a single bond,
(2) alkylene,
(3) alkenylene, or
(4) alkynylene.

2. A compound of claim 1 wherein
R¹ is H;
R² is
(a) heteroaryl optionally substituted with one to three groups T¹, T², T^{3;}
(b) aryl substituted with one to three groups T¹, T², T³ independently selelected from optionally substituted heteroaryl, cyano, C(O)ₜT⁶, S(O)ₜN(T⁹)T⁶, halo alkyl, and haloalkyl); or
(c) quinolyl, quinazolinyl, cinnolinyl, isoqinolinyl, or phthalazinyl any of which may be optionally substituted with one to three groups T¹, T², T³;
Z is NR³R⁴, or OR⁴;
R³ is H or alkyl, cycloalkyl;
R⁴ is alkyl or (aryl)alkyl either of which may be optionally independently substituted with one to three groups T^{1a}, T^{2a} or T^{3a};
or R³ and R⁴ may be taken together with the nitrogen atom to which they are attached to form a heterocyclo ring optionally independently substituted with one to three groups T^{1a}, T^{2a} or T^{3a};
R⁵ is
(a) hydrogen, or cyano;
(b) alkyl, alkenyl, alkynyl, cycloalkyl, (cycloalkyl)alkyl, aryl, (aryl)alkyl, heterocyclo, (heterocyclo)alkyl, heteroaryl or (heteroaryl)alkyl, any of which may be optionally independently substituted one to three groups T^{1b}, T^{2b} or T^{3b}; or
(c) —C(O)R⁶, —C(O)OR⁶, —C(O)—C(O)OR , or —SO₂R^{6a};
R⁶ is H, alkyl, alkenyl, —NR^{3b}R^{4b}, heterocylco (heterocyclo)alkyl, (hydroxy)alkyl, (alkoxy)alkyl, (aryloxy)alkyl, (NR^{3b}R^{4b})alkyl, heteroaryl, (heteroaryl)alkyl, aryl or (aryl)alkyl, any of which may be optionally independently substituted with one to three groups T^{1b}, T^{2b} or T^{3b}; and
R^{6a} is alkyl, alkenyl, —NR^{3b}R^{4b}, heterocylco (heterocyclo)alkyl, (hydroxy)alkyl, (alkoxy)alkyl, (aryloxy)alkyl, (NR^{3b}R^{4b})alkyl, heteroaryl, (heteroaryl)alkyl, aryl or (aryl)alkyl, any of which may be optionally independently substituted where valance allows with one to three groups T^{1b}, T^{2b} or T^{3b}.

3. A compound of claim 2 wherein
R² is
(a) thiazolyl optionally substituted with one to three groups T¹, T², T³, where T¹, T², T³ are independently H, alkyl, haloalkyl, halo, heteroaryl, C(O)ₜT⁶, OT⁶, —T⁴NT⁷T⁸
(b) phenyl substituted at the para position with T¹, and optionally further substituted with groups T² and T³ where
T¹ is optionally substituted heteroaryl, cyano, C(O)ₜT⁶, or S(O)ₜN(T⁹)T⁶, and
T² and T³ are independently H, heteroaryl, cyano, C(O)ₜT⁶, S(O)ₜN(T⁹)T⁶, halo alkyl, and haloalkyl) or
(c) quinol-6-yl, quinazolin-6-yl, cinnolin-6-yl, isoquinol-6-yl, or phthalazin-6-yl, any of which may be optionally substituted with one to three groups T¹, T², T³;
Z is NR³R⁴;
R³ is H or alkyl, cycloalkyl;
R⁴ is (aryl)alkyl optionally independently substituted with one to three groups T^{1a}, T^{2a} or T^{3a}, where T^{1a}, T^{2a} or T^{3a} are independently OT⁶, S(O)ₜT⁶ or S(O)ₜN(T⁹)T⁶;
or R³ and R⁴ may be taken together with the nitrogen atom to which they are attached to form piperidyl, piperazinyl, or morpholinyl any of which may be optionally independently substituted with one to three groups T^{1a}, T^{2a} or T^{3a} where T^{1a}, T^{2a} or T^{3a} are independently H, hydroxy, oxo, and —C(O)ₜT⁶;
R⁵ is
(a) hydrogen, or cyano;
(b) alkyl, alkenyl, (cycloalkyl)alkyl, (aryl)alkyl, or (heteroaryl)alkyl any of which may be optionally independently substituted one to three groups T^{1b}, T^{2b} or T^{3b} wherein T^{1b}, T^{2b} or T^{3b} are independently H, cyano, —OT⁶, and —S(O)ₜT⁶; or
(c) —C(O)R⁶, —C(O)OR⁶, —C(O)—C(O)OR⁶, or —SO₂R^{6a};
R⁶ is H, alkyl, alkenyl, —NR^{3b}R^{4b}, heterocylco, (heterocyclo)alkyl, (hydroxy)alkyl, (alkoxy)alkyl, (aryloxy)alkyl, (NR^{3b}R^{4b})alkyl, any of which may be optionally independently substituted one to three groups T^{1b}, T^{2b} or T^{3b} where T^{1b}, T^{2b} or T^{3b} are independently H, alkyl, —C(O)ₜH, —C(O)ₜT⁶, —OC(O)T⁶, —OH, —OT⁶, and —S(O)ₜT⁶; and
R^{6a} is H, alkyl, alkenyl, —NR^{3b}R^{4b}, heterocylco, (heterocyclo)alkyl, (hydroxy)alkyl, (alkoxy)alkyl, (aryloxy)alkyl, (NR^{3b}R^{4b})alkyl, any of which may be optionally independently substituted with one to three groups T^{1b}, T^{2b} or T^{3b} where T^{1b}, T^{2b} or T^{3b} are independently H, alkyl, —C(O)ₜH, —C(O)ₜT⁶, —OC(O)T⁶, —OH, —OT⁶, or —S(O)ₜT⁶.

4. A compound of claim 1 having formula (IIa), or (IIb) 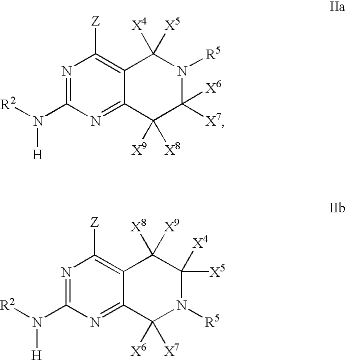
wherein:
R² is 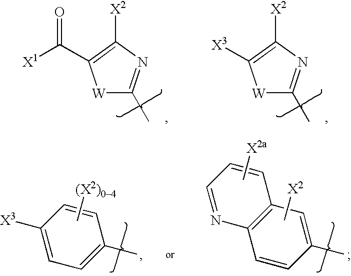
W is O or S;
X¹ is NHT⁸ or OT⁶;
X² and X^{2a} are independently hydrogen, halo, OT⁶, or alkyl; and
X³ is optionally substituted heteroaryl, cyano, C(O)ₜT⁶, or S(O)ₜN(T⁹)T⁶;
X⁴, X⁵, X⁶ and X⁷ are independently H, T⁶, OT⁶, or NT⁷ T⁸, or X⁴ and X⁵ or X⁶ and X⁷ may be taken together to be a carbonyl group; and
X⁸ and X⁹ are independently H, T⁶, OT⁶, or NT⁷T⁸.

5. A compound of claim 1 having formula IIIa, IIIb or IIIc 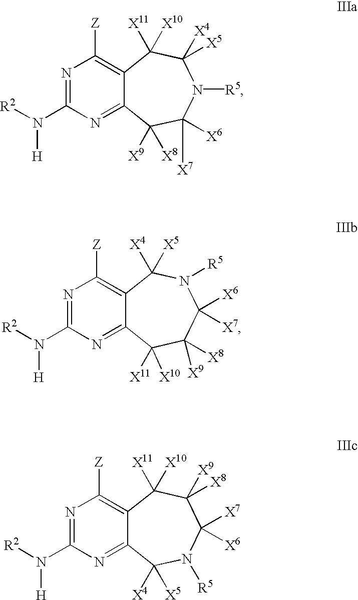
wherein
R² is 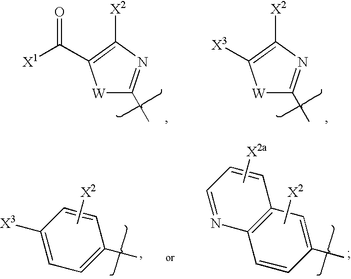
W is O or S;
X¹ is NHT⁸ or OT⁶;
X² and X^{2a} are independently hydrogen, halo, OT⁶, or alkyl;
X³ is optionally substituted heteroaryl, cyano, C(O)ₜT⁶, or S(O)ₜN(T⁹)T⁶;
X⁴, X⁵, X⁶ and X⁷ are independently hydrogen, T⁶, OT⁶, or NT⁷T⁸, or X⁴ and X⁵, or X⁶ and X⁷ may be taken together to be a carbonyl group; and
X⁸, X⁹ X¹⁰, and X¹¹ are independently hydrogen, T⁶, OT⁶, or NT⁷T^{8.}

6. A compound of claim 1 having formula IV 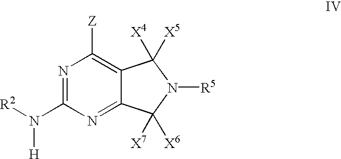
wherein:
R² is 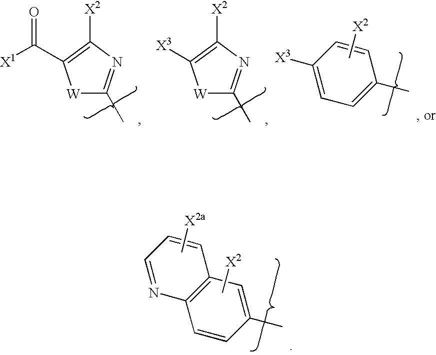
W is O or S;
X¹ is NHT⁸ or OT⁶.
X² is hydrogen, halo, OT⁶, or alkyl.
X³ is optionally substituted heteroaryl, cyano, C(O)ₜT⁶, or S(O)ₜN(T⁹)T⁶;
X⁴, X⁵, X⁶ and X⁷ are independently H, T⁶, OT⁶, or NT⁷T⁸, or X⁴ and X⁵, or X⁶ and X⁷ may be taken together to be a carbonyl group.

7. A pharmaceutical composition comprising at least one compound of claim 1.

8. A method of treating T-cell mediated diseases which comprises administering an effective amount of at least one compound of claim 1 to a patient in need thereof.

9. A method of claim 8 wherein said T-cell mediated disorder is transplant rejection.

10. A method of claim 8 wherein said T-cell mediated disorder is graph verses host disease.

11. A method of claim 8 wherein said T-cell mediated disorder is rheumatoid arthritis.

12. A method of claim 8 wherein said T-cell mediated disorder is multiple sclerosis.

13. A method of claim 8 wherein said T-cell mediated disorder is juvenile diabetes.

14. A method of claim 8 wherein said T-cell mediated disorder is asthma.

15. A method of claim 8 wherein said T-cell mediated disorder is inflammatory bowel disease.

16. A method of claim 8 wherein said T-cell mediated disorder is ischemic or reperfusion injury.

17. A method of claim 8 wherein said T-cell mediated disorder is cell proliferation.

18. A method of claim 8 wherein the T-cell mediated disorder is psoriasis.

19. A pharmaceutical composition of claim 7 further comprising at least additional therapeutic agent selected from PDE 4 inhibitors, NSAIDs, COX-2 inhibitors, TNF-α inhibitors, beta-2 agonists, anti-cholinergic agents, and steriods.
